(19) United States
(12) Patent Application Publication
(10) Pub. No.: US 2015/0147791 A1
(43) Pub. Date: May 28, 2015

(54) METHANE-TO-ACETATE PATHWAY FOR PRODUCING LIQUID BIOFUELS AND BIORENEWABLES

(71) Applicant: The Penn State Research Foundation, University Park, PA (US)

(72) Inventors: James Gregory Ferry, University Park, PA (US); Costas D. Maranas, University Park, PA (US); Thomas Keith Wood, University Park, PA (US)

(21) Appl. No.: 14/550,547

(22) Filed: Nov. 21, 2014

Related U.S. Application Data
(60) Provisional application No. 61/907,086, filed on Nov. 21, 2013.

(51) Int. Cl. C12P 7/54; C12N 9/10; C12P 7/14

(52) U.S. Cl. CPC C12P 7/54; C12P 7/14; C12N 9/13; C12Y 208/04001
USPC 435/140; 435/243; 435/162; 435/160

(57) ABSTRACT

The engineering of a pathway converting CH₄ to acetate and eventually to liquid fuels is disclosed. The engineered pathway involves an engineered reversal of the natural pathway for acetate conversion to CH₄ by microbes and coupling the engineered pathway to existing and future technologies for microbial or chemical conversion of acetate to liquid fuels. In one aspect, methods for producing modified pathways and/or microbes are provided. In another aspect engineered microbes, including Methanosarcina acetivorans, that incorporate the engineered pathway are provided, which can mediate conversion of CH₄ to acetate for ultimate conversion to liquid fuels. In a further aspect, specific modifications to the components of the pathway are provided.

### PRIORITY STATEMENT

This application claims priority to U.S. Provisional Patent Application No. 61/907,086, filed Nov. 21, 2013, hereby incorporated by reference in its entirety.

### FIELD OF THE INVENTION

The present invention relates to biofuels and biorenewables. More particularly, but not exclusively, the present invention relates to engineering a methane-to-acetate pathway for producing liquid biofuels.

### BACKGROUND

The United States has vast reserves of methane; however, many of these methane reserves are remote and a significant amount of the methane is released into the atmosphere (this is detrimental since methane is a potent greenhouse gas). There is a need to convert these resources easily, reliably, and efficiently into liquid fuels.

### SUMMARY OF THE INVENTION

Therefore, it is a primary object, feature, or advantage of the present invention to improve over the state of the art.

It is a further object, feature, or advantage of the present invention to convert methane reserves into liquid fuels.

It is a still further object, feature, or advantage of the present invention to reduce greenhouse gas from the atmosphere.

It is another object, feature, or advantage of the present invention to provide for conversion of methane gas into liquid fuels in a manner that is easy, reliable, and efficient.

One or more of these and/or other objects, features, or advantages of the present invention will become apparent from the specification and claims that follow. No single embodiment need exhibit each or every object, feature, or advantage as it is contemplated that different embodiments or aspects of the invention may have different objects, features, or advantages.

According to one aspect, the present invention provides compositions and methods for creating metabolically-engineered microbes that converts methane gas to a biofuel such as ethanol. For example, the activity of methylreductase of M. acetivorans can be reversed to create a pathway that converts methane to acetate. Moreover, the invention includes methods for converting acetate to biofuels, such as but not limited to, ethanol or butane, for example by: (i) further engineering the pathway to accommodate supplemental reductant (CO and/or electricity) or (ii) coupling M. acetivorans with pure cultures or consortia of anaerobes thermodynamically assisted with H₂ or CO.

### BRIEF DESCRIPTION OF THE DRAWINGS

FIG. 1 illustrates the global carbon cycle. The top part depicts steps in aerobic zones dependent on O₂ and the bottom graph depicts steps in O₂-free anaerobic zones.

FIG. 2 illustrates cofactors and coenzymes utilized in the aceticlastic pathway of methanogenesis.

FIG. 3 illustrates the acetotrophic pathway of the marine isolate M. acetivorans.

FIG. 4 illustrates an expression vector pES1. Panel A shows the details of the cloning sites and panel B shows the entire plasmid. Vector contains genes for selection by ampicillin resistance in E. coli and puromycin resistance in M. acetivorans. The origins of replication are OriR6K and On pC2A for E. coli and M. acetivorans, respectively. Downstream of the cdh promoter is a multiple cloning site flanked by sequence for 6× His tags and the T7 tag for optional protein fusions to aid in protein purification. An N-terminal 6× His fusion may be cleaved by digestion with thrombin using the thrombin digestion site. After the cdh promoter are an archaeal ribosomal binding site (aRBS) and a bacterial ribosomal binding site (bRBS).

FIG. 5 illustrates steps of reconstruction pipeline.

FIG. 6 illustrates a pictorial illustration of OptCom.

FIG. 7 illustrates a multi-species microbial system design.

FIG. 8 illustrates the Iterative Protein Redesign & Optimization Workflow.

FIG. 9 illustrates OptZyme Validation on a Benchmark System for (left) K_{M}, (center) k_{cat}/K_{M}, and (right) k_{cat}

FIG. 10 illustrates the alpha subunit (TouA) of ToMO shows the locations of the important TouA-hydroxylase residues. The diiron center is shown in orange. Many of the beneficial positions such as 1100 (blue), A101 (yellow), E103 (orange), A107 (red), and A110 (green) are located on the TouA α-helix (highlighted in red). Other beneficial positions such as T201 (purple), F205 (light blue), and E214 (pink) are located in the TouA E-helix (highlighted in yellow). Position Q141 and M180 are shown in light pink and dark pink, respectively.

FIG. 11 illustrates a synthetic dispersal metabolic circuit and microfluidic device (a) The two E. coli cell types communicate by using the LasI/LasR QS module of P. aeruginosa. In the disperser cell, the LasI protein (autoinducer synthase) is constitutively produced and synthesizes the quorum-sensing signal 3oC12HSL. 3oC12HSL freely diffuses into the initial colonizer cell and binds LasR which induces biofilm dispersal protein BdcAE50Q by activating the lasI promoter. The biofilm dispersal protein Hha13D6 in the disperser cell is induced upon adding IPTG. (b) The novel microfluidic device is shown with its two PDMS layers, a bottom layer with a diffusive-mixer and eight microchambers, and a top layer containing a second diffusive mixer and the pneumatic elements to control microvalves.

FIG. 12 illustrates dispersal of dual-species biofilms using quorum sensing. (a) An initial colonizer biofilm (red, BdcAE50Q⁺, LasR⁺; E. coli hha/pBdcAE50Q-rfp-lasR) was developed for 9 h, then disperser cells (green, Hha13D6⁺, LasI⁺; E. coli hha/pHha13D6-gfp-lasI) were seeded for 5 h to form both initial colonizer and disperser biofilms. After 44 h, 2 mM of IPTG was added for an additional 18 h to remove the disperser biofilm. (b) Initial colonizer biofilms (BdcAE50Q⁺, LasR⁺) was developed for 9 h, then control disperser cells which lack LasI (green, Hha13D6⁺, LasI⁻; E. coli hha/pHha13D6-gfp) were seeded for 5 h to form both initial colonizer and control disperser biofilms. After 40 h, 2 mM of IPTG was introduced for an additional 20 h to try to disperse the control disperser biofilms which lack lasI.

FIG. 13 illustrates the proposed reversal of the acetate-to-methane pathway in M. acetivorans. H₄SPT, tetrahydrosarcinapterin; Fd, ferredoxin.

FIG. 14 illustrates ferredoxin:CoM-S—S-CoB oxidoreductase activity in the soluble fraction of acetate-grown M. acetivorans. Symbols: (♦) complete reaction, (▪) minus ferredoxin, (▴) minus soluble fraction.

FIG. 15 illustrates a proposed pathway in M. acetivorans

FIG. 16 illustrates detection of coenzyme B with 7-nitro-2,3-dihydro-1H-cyclopenta[b]chromen-1-one⁴⁰.

FIG. 17(A-C) shows reversibility of Mcr and Hdr. A shows a schematic representation of the analysis. B shows the reaction vials: Silver cap, N2 atmosphere; blue cap, methane atmosphere. C shows quantification of the color change observed in the vials.

FIG. 18 shows NMR analyses showing recovery of ¹³C in methylated compounds after incubation of ¹³CH₄ with resting cell suspensions of methanol-grown Methanosarcina acetivorans.

FIG. 19(A-B) shows the natural acetate-to-methane (A) and engineered methane-to-acetate (B) pathways.

FIG. 20 shows reduction of FldII with reduced Fd.

FIG. 21 shows reduction of Etp and PolyFd with reduced Fd. CO was the electron donor to Cdh reducing Fd.

FIG. 22 shows reconstitution of iron-sulfur clusters in Etp and PolyFd.

FIG. 23 shows Western blot analysis of Mre A,B,C and D with antisera directed towards MreA. The gel was loaded with 100 and 200 nanograms of proteins.

FIG. 24 (A-D) shows phase-contrast micrographs of growth of M. acetivorans C2A/pES1-MATmcr grown in the presence of 100% methane and 0.1 mM FeCl3. A & B, Culture at day 0 (post-inoculation). C & D, cells at day 30. Scale bar denotes 5 μm.

FIG. 25 shows total protein content for methane-grown, engineered M. acetivorans C2A/pES1-MATmcr cells. 1, Culture grown in methane and 0.1 mM Fe³⁺. 2, Negative control (inoculated growth medium where growth is absent). 3, Culture grown in methanol (the preferred substrate for M. acetivorans).

FIG. 26(A-B) shows HPLC chromatograms showing the presence of formate and acetate in cultures of M. acetivorans C2A/pES1-MATmcr grown on methane after 71 days of incubation. A, Chromatogram from culture grown on methane and 1 mM FeSO₄ with an acetate peak area corresponding to a concentration of 5 mM acetate. B, Chromatogram from the negative control without signs of growth with an acetate peak area corresponding to a concentration of 0.1 mM acetate.

FIG. 27 shows alignment of methanogenic Mcrs and a single ANME Mcr. Alignment between the structures was performed by using BLAST, followed by minimizing the RMSD between the backbone atoms of the structures. All alignments used the coordinates of ANME-1 Mcr (the lone cystallized ANME Mcr) as reference. The four structures are largely similar despite the fact that one catalyzes the reaction in the methanotrophic direction. Methanogenic Mcrs are shown in various shades of red (Red: M. kandleri; Pink: M. marburgensis; Ruby: M. barkeri), and the ANME-1 Mcr is shown in blue. The four enzymes are nearly identical close to the active site (indicated by F430 shown in yellow).

FIG. 28 shows contacts made by the top mutant of Table 5 (left) and wild-type (right) with the modified portion of F430. The active site of ANME-1 Mcr is shown here. The unmodified cofactor is shown in green, the enzyme in cyan, mutation M78R in orange, position 157 (D for mutant 1, H for wild-type) in pink, and mutation V419K is shown in black. Three different orientations are provided for the active site, with the mutant structure on the left and the corresponding wild-type structure on the right. The C172 carbon atom and its two bonded hydrogen atoms are shown in a space-filling representation (in green), and all atoms within 3.5 Å of these three atoms are also shown in a space-filling representation.

FIG. 29 shows gene lethality predictions by iMAC865. Comparison of in vivo and in silico results for gene lethality predictions for growth on methanol and acetate after the implementation of the R-GPR approach.

### DETAILED DESCRIPTION

### Definitions

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4,5, etc.). For any method disclosed herein that includes discrete steps, the steps may be conducted in any feasible order. And, as appropriate, any combination of two or more steps may be conducted simultaneously.

As used herein, the term “biofuel” is intended to mean a liquid fuel produced using a microbial biology based process. Thus, the term “biofuel” includes fuels made from fossil methane (e.g. natural gas, coal bed methane), as well as fuels where the carbon feedstock is recently from a biological source (e.g. biorenewables).

As used herein, the term “microbe” refers to any microscopic organism, which may be a single cell or multicellular organism. The term is understood to include all of the bacteria and archaea and almost all the protozoa. They also include some members of the fungi, algae.

“Methanogens” refers to microorganisms that produce methane as a metabolic byproduct in anoxic conditions. Methanogens are understood to include any microorganism that possesses the cellular machinery for producing methane, including those microorganisms with endogenous systems and microorganisms expressing heterologous systems. Methanogens include bacteria and fungi, but are most commonly classified as archaea. They are common in wetlands, where they are responsible for marsh gas, and in the digestive tracts of animals such as ruminants and humans. Only two genera of archea methanogens, Methanosarcina (Msr.) and Methanosaeta (Mse.), contain species that produce methane by the “aceticlastic” pathway . Microbes that produce methane by the “aceticlastic” pathway, including but not limited to Msr. And Mse. microbes, are referred to herein as “acetate-utilizing” or “ aceticlastic”, which are used interchangeably. Methanosaeta (previously Methanothrix) species have a higher affinity for acetate than Methanosarcina and dominate in methanogenic habitats with low concentrations of acetate. The biochemistry of the aceticlastic pathway has been investigated primarily in the freshwater Methanosarcina species Msr. barkeri, Msr. mazei and Msr. thermophila and the marine isolate Msr. acetivorans.

“Methylreductase” (“Mcr”) refers to a group of enzymes, including coenzyme-B sulfoethylthiotransferase, also known as methyl-coenzyme M reductase or most systematically as 2-(methylthio)ethanesulfonate:N-(7-thioheptanoyl)-3-O-phosphothreonine S-(2-sulfoethyl)thiotransferase, that catalyze the final step in the formation of methane. Mcr refers to any enzyme that combines the hydrogen donor coenzyme B and the methyl donor coenzyme M. Mcr are most commonly expressed by methanogens.

As used herein, “mutation” includes reference to alterations in the nucleotide sequence of a polynucleotide, such as for example a gene or coding DNA sequence (CDS), compared to the wild-type sequence. The term includes, without limitation, substitutions, insertions, frameshifts, deletions, inversions, translocations, duplications, splice-donor site mutations, point-mutations or the like.

As used herein, “nucleic acid” includes reference to a deoxyribonucleotide or ribonucleotide polymer in either single-or double-stranded form, and unless otherwise limited, encompasses conservatively modified variants and known analogues having the essential nature of natural nucleotides in that they hybridize to single-stranded nucleic acids in a manner similar to naturally occurring nucleotides (e.g., peptide nucleic acids).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, “polynucleotide” includes reference to a deoxyribopolynucleotide, ribopolynucleotide, or conservatively modified variants; the term may also refer to analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art.

The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms also may apply to conservatively modified variants and to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not always entirely linear. For instance, polypeptides may be branched as a result of ubiquitization, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may affect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to another nucleic acid sequence or other biologics. When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. The term “sequence” refers to an amino acid or nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value there between or thereabove), preferably between about 100 and 1,000 nucleotides in length (or any integer there between), more preferably between about 200 and 500 nucleotides in length.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination there between, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively.

Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression.

As used herein, the terms “coding region” and “coding sequence” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

A polynucleotide that includes a coding region may include heterologous nucleotides that flank one or both sides of the coding region. As used herein, “heterologous nucleotides” refer to nucleotides that are not normally present flanking a coding region that is present in a wild-type cell. For instance, a coding region present in a wild-type microbe and encoding a Mcr polypeptide is flanked by homologous sequences, and any other nucleotide sequence flanking the coding region is considered to be heterologous. Examples of heterologous nucleotides include, but are not limited to regulatory sequences. Typically, heterologous nucleotides are present in a polynucleotide disclosed herein through the use of standard genetic and/or recombinant methodologies well known to one skilled in the art. A polynucleotide disclosed herein may be included in a suitable vector.

Conservatively modified variants refer to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid.

One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made.

Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T); 2) Aspartic acid (D), Glutamic acid (E); 3) Asparagine(N), Glutamine (Q); 4) Arginine (R), Lysine (K); 5) Isoleucine(I), Leucine (L), Methionine (M), Valine (V); and 6)Phenylalanine (F), Tyrosine (Y), Tryptophan (W). See also, Creighton (1984) Proteins W. H. Freeman and Company. The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

As used herein “full-length sequence” in reference to a specified polynucleotide or its encoded protein means having the entire amino acid sequence of a native (nonsynthetic), endogenous, biologically active form of the specified protein. Methods to determine whether a sequence is full-length are well known in the art including such exemplary techniques as northern or western blots, primer extension, S 1 protection, and ribonuclease protection. Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

### Overview

Engineered pathways are provided for converting methane (CH₄) to liquid fuels using a two-phase approach. Phase I involves an engineered reversal of the natural pathway for acetate conversion to CH₄, such as by Methanosarcina acetivorans. Phase II involves converting the acetate to liquid fuels via CO oxidation, co-culture with acetate utilizing species or non-biological processes. It is important to note that naturally occurring cultures produce ethanol from acetate utilizing H₂ as a reductant. Phase I has three significant advantages: (i) the pathway conserves all electrons in CH₄, (ii) consumes CO₂ and (iii) produces a C—C bond. The choice of M. acetivorans is appropriate as: (i) trace CH₄ oxidation has been documented, (ii) a novel methanogenic pathway (the first and only) has been engineered in M. acetivorans, (iii) the genome is sequenced and a well-documented robust genetic system is available, (iv) the first, and only, over-production of an active methanogen metalloenzyme was accomplished in M. acetivorans, (v) the pathway for acetate formation from relevant pathway intermediates has been elucidated, (vi) the enzymology required for potential CH₄ conversion to acetate is well-characterized, (vii) novel enzymes and physiologies to reverse engineer the acetate-to-CH₄ pathway are in-hand hand and (viii) a molecular understanding of gene regulation in the acetate-to-CH₄ pathway is well underway. After reversing the acetate-to-methane pathway, methane may be converted to ethanol by supplying a reductant (CO or electricity) to M. acetivorans that has been further engineered to express three genes for ethanol production from acetate. Alternatively, the engineered M. acetivorans may be coupled with cultures converting acetate to ethanol assisted with CO or H₂.

Opportunity of the Marcellus Shale. Initiated in 2004, horizontal drilling has made the methane resources of the Marcellus Shale economical to be tapped through hydraulic fracturing. The Marsellus Shale is a 54,000 square mile black shale formation that is up to 10,000 feet below primarily Pennsylvania and New York and holds up to 516 trillion cubic feet of natural gas; for Pennsylvania, the recoverable gas is worth more than $500B. Based on these and other reserves, this creates an exciting time in which the U.S. can think of energy independence. Unfortunately, methane is released in significant quantities as a greenhouse gas in some cases, and it is difficult to transport in remote areas. Also, liquid biofuels are easier to use for our existing automotive infrastructure. Therefore, it would be advantageous to in situ convert methane to a liquid fuel. This application aims to accomplish this through biological means by engineering a microbe such as Methanosarcina acetivorans to convert methane gas for use as a liquid fuel. In particular, M. acetivorans may be engineered to reverse the natural pathway for conversion of acetate to methane and then biological and/or non-biological methods may be used for converting the acetate to liquid fuels.

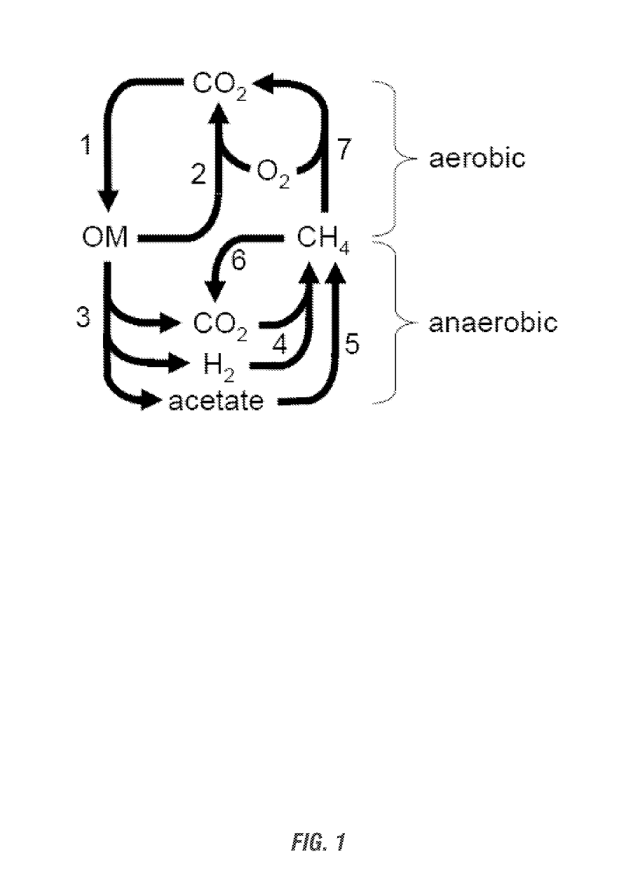

Microbiology of the natural pathway converting acetate to methane. Methane is the end product of the decomposition of complex organic matter in diverse O₂-free (anaerobic) environments, producing nearly one billion metric tons of methane each year. The process is an essential link in Earth's carbon cycle (FIG. 1). In the cycle, CO₂ is fixed into complex organic matter by photosynthesis (step 1) and in aerobic zones is oxidized back to CO₂ by O₂-requiring microbes (step 2). A portion of the organic matter is deposited in a variety of anaerobic environments where diverse anaerobes decompose the organic matter (step 3) to products that are substrates for methane-producing species (steps 4 and 5). Some of the methane is utilized by sulfate- or nitrate-reducing anaerobic methane oxidizers (step 6) and the remainder escapes into aerobic habitats where it is oxidized to CO₂ by O₂-requiring methylotrophic microbes (step 7).

Approximately one-third of the methane produced in Earth's biosphere is generated by the reduction of CO₂ with electrons derived from the oxidation of H₂ (FIG. 1, step 4):

CO₂+4H₂43 CH₄+2H₂O [Eq. 1]

The remaining two-third originates from the methyl group of acetate (FIG. 1, step 5) by the “aceticlastic” pathway:

CH₃COO⁻+H⁺→CH₄30 CO₂ [Eq. 2]

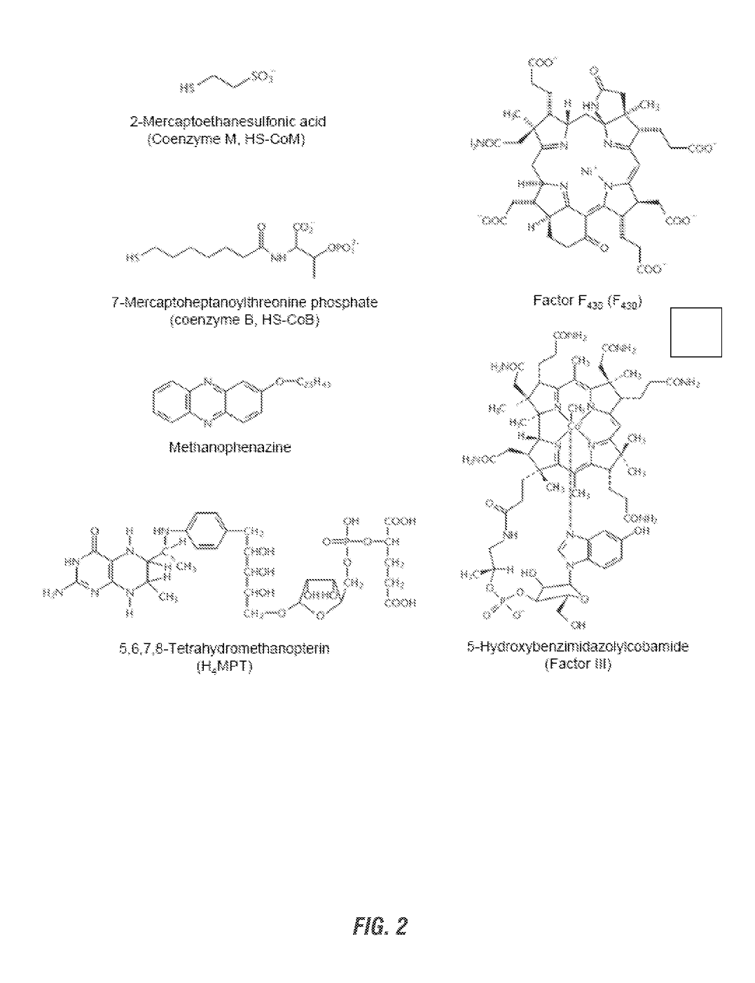

Pathway converting acetate to methane. FIG. 2 shows the cofactors and coenzymes involved in the aceticlastic pathway. Factor III, tetrahydromethanopterin (H₄SPT), coenzyme M (HS-CoM) and factor F₄₃₀ (F₄₃₀) are involved in transfer of the methyl group of acetate to methane. Coenzyme M (HS-CoM) carries a methyl group attached to the sulfur atom. F₄₃₀ is a nickel-containing corphinoid tetrapyrrole that binds a methyl group to the metal atom. CoB-SH and methanophenazine (MP) are electron carriers.

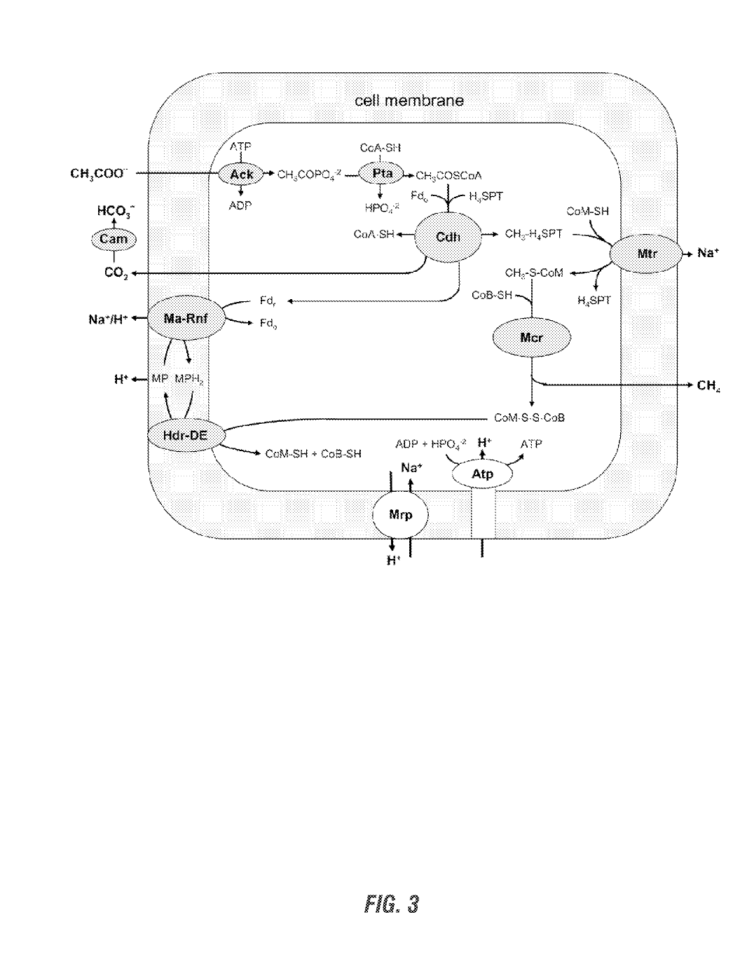

Biochemical and bioinformatic evidence indicates that the core methyl transfer steps leading from the methyl group of acetate to methane are similar in all freshwater and marine species (FIG. 3). Reactions involved in transfer of the methyl group from acetate to produce CH₃—H₄SPT (Eqs. 3-4) are catalyzed by enzymes with homologs widely dispersed in the Bacteria domain.

CH₃COO⁻+ATP→CH₃CO₂PO₃⁻²+ADP [Eq. 3]

CH₃CO₂PO₃⁻²+HS-CoA→CH₃COSCoA+Pi [Eq. 4]

CH₃COSCoA+H₄SPT+H₂O+Fdₒ→CH₃—H₄SPT+FdᵣCO₂+HS-CoA [Eq. 5]

The structure and function of the enzymes catalyzing these reactions have been investigated in considerable detail, the understanding of which has impacted the broader field of prokaryotic biology in view of the fact that paralogs are wide-spread in diverse anaerobes from the Bacteria domain. Acetate kinase (FIG. 3, Ack) and phosphotransacetylase (FIG. 3, Pta) catalyze reactions (Eqs. 3 and 4) that together activate acetate to acetyl-CoA which is the substrate for CO dehydrogenase/acetyl-CoA (FIG. 3, Cdh), the central enzyme in the pathway catalyzing the reaction shown in Eq. 5. In Methanosaeta species, acetate is converted to acetyl-CoA in one reaction (Eq. 6) catalyzed by acetyl coenzyme A synthetase.

CH₃COO⁻+ATP+CoASH→CH₃COSCoA+AMP+PiPi [Eq. 6]

The Cdh cleaves the C—C and C—S bonds of acetyl-CoA (Eq. 5) yielding methyl and carbonyl groups. The methyl group is transferred to H₄SPT for eventual conversion to methane and the carbonyl group is oxidized to CO₂ with the electrons transferred to a 2x[4Fe-4S] ferredoxin. In addition to aceticlastic methanogens, diverse anaerobes from the Bacteria domain utilize Cdh in energy-yielding pathways generating acetyl-CoA with CoA-SH, a methyl group and CO₂ plus a pair of electrons. The acetyl-CoA is further metabolized to acetate catalyzed by phosphotransacetylase and acetate kinase producing ATP. The Cdh from methanogens has been biochemically characterized from both Methanosarcina and Methanosaeta species. Most structure and function studies have been performed with the enzymes from Msr. thermophila and Msr. barkeri. In both species the enzyme is a complex comprised of five-subunits (α,β,γ,δ,ε) resolvable by detergent treatment into a Ni/Fe—S component (α and ε subunits), a Co/Fe—S component (γ and δ subunits), and the β subunit. The resolved Ni/Fe—S component catalyzes the reversible oxidation of CO to CO₂ utilizing a 2x[4Fe-4S] ferredoxin as the redox partner consistent with a role for this component in oxidizing the carbonyl group of acetyl-CoA and reducing ferredoxin. The Co/Fe—S component of the Cdh from Methanosarcina species contains Factor III and is involved in transfer of the methyl group of acetyl-CoA to H₄SPT. The Co/Fe—S component also contains a [4Fe-4S] cluster with a midpoint potential at pH 7.8 of −502 mV, which is nearly isopotential with the Co²⁺/Co¹⁺ couple and likely serves as the direct electron donor. The cdhD and cdhE genes which encode the δ and γ subunits have been cloned and sequenced. The CdhE sequence contains a four-cysteine motif with the potential to bind a 4Fe-4S cluster. Further, CdhE overproduced in E. coli contains the cluster and a corrinoid cofactor with the benzimidazole base in the base-off configuration. The results are consistent with a role for the γ subunit in transfer of the methyl group to H₄SPT.

The conversion of CH₃—H₄SPT to methane is common to all methanogenic pathways and requires three reactions catalyzed by an eight-subunit, membrane-bound, CH₃—H₄SPT:coenzyme M methyltransferase (MtrA-H) (Eq. 7), CH₃-CoM methylreductase (McrABC) (Eq. 8) and heterodisulfide reductase (HdrDE) (Eq. 9).

CH₃-THMPT+HS-CoM→CH₃—S-CoM+THMPT [Eq. 7]

CH₃—S-CoM+HS-CoB→CoMS—SCoB+CH₄ [Eq. 8]

CoM-S—S-CoB+2e⁻+2H⁺→HS-CoB+HS-CoM [Eq. 9]

The methyltransferase (FIG. 3, Mtr) has been characterized from acetate-grown cells of Msr. mazei and other methanoarchaea. The enzyme, comprised of eight non-identical subunits couples the exergonic methyl transfer to generation of a sodium ion gradient (high outside the cytoplasmic membrane) postulated to drive various energy-requiring reactions. The reaction shown in Eq. 8 is catalyzed by methyl-CoM methylreductase (FIG. 3 Mcr) that contains nickel in coenzyme F₄₃₀. HS-CoB is the electron donor that when oxidized forms a disulfide bond with HS-CoM (CoM-S—S-CoB) in addition to methane. A two-subunit heterodisulfide reductase (FIG. 3, Hdr) identified in acetate-grown cells of Msr. thermophila and Msr. barkeri is consistent with a role for catalysis of the disulfide bond of CoM-S—S-CoB and regenerating the active sulfhydryl forms of the coenzymes (Eq. 9).

The heterodisulfide CoM-S—S-CoB is the terminal electron acceptor of a membrane-bound electron transport chain coupled to formation of an electrochemical ion gradient driving ATP synthesis. The “archaeal” A₁A₀-type ATP synthase is abundant in acetate-grown Msr. acetivorans and Msr. mazei consistent with a role in ATP synthesis. Freshwater Methanosarcina species utilize a membrane-bound reduced ferredoxin (Fdᵣ):CoM-S—S-CoB oxidoreductase system involving the production and consumption of H₂. However, Msr. acetivorans, a marine species, has evolved a mechanism for oxidizing ferredoxin and reducing CoM-S—S-CoB that does not involve H₂ (FIG. 3). Based on quantitative proteomic and biochemical analyses, it is proposed that a homolog of Rnf (FIG. 3B, Ma-Rnf) oxidizes ferredoxin and that methanophenazine mediates electron transfer between reduced Ma-Rnf and the heterodisulfide reductase HdrDE. Rnf was first discovered in Rhodobacter capsulatus and shown to be a six-subunit membrane-bound electron transfer complex with homologs wide spread in the Bacteria domain that are proposed to couple electron transport to the generation of a Na⁺ gradient. The six subunits of the Msr. acetivorans Ma-Rnf complex are encoded in a transcriptional unit with two additional flanking ORF's, one of which encodes a cytochrome c. It is proposed that the Ma-Rnf complex and cytochrome c function to transport electrons from ferredoxin to methanophenazine coupled to generation of a sodium gradient (high outside the membrane). Subunits of a seven-subunit Na⁺/H⁺ antiporter (FIG. 3B, Mrp) are at least 30-fold more abundant in acetate vs. methanol-grown Msr. acetivorans suggesting a role for this complex to adjust the Na⁺/H⁺ ratio optimal for the Na⁺ and H⁺-dependent A₁A₀ ATP synthase (FIG. 3).

The conversion of acetate to CH₄ and CO₂ provides only a marginal amount of energy available for ATP synthesis (ΔG°′=−36 kJ/CH₄). A calorimetric and thermodynamic analysis of Msr. barkeri grown with acetate suggests a retarding effect of the positive enthalpy change on the driving force of growth that is overcompensated by a large positive entropy change resulting from the conversion of acetate to only gaseous products. Since both the enthalpy and the entropy increases are due in part to transition of CH₄ and CO₂ into the gaseous phase, it is proposed that a carbonic anhydrase (FIG. 3, Cam) facilitates removal of CO₂ from the cytoplasm by converting CO₂ to membrane-impermeable HCO₃⁻ outside the cell membrane (Eq. 10).

CO₂+H₂O→HCO₃⁻+H⁺ [Eq. 10]

The synthesis of Cam is up-regulated in Msr. thermophila, Msr. acetivorans and Msr. mazei when switched from growth on methanol to growth on acetate consistent with a role during growth on acetate. Cam from Msr. thermophila is the archetype of an independently-evolved class of carbonic anhydrases (γ class) and is the first carbonic anhydrase shown to function with iron in the active site. This provides an overview of the native methane utilization pathways in archaea. Enzyme engineering techniques, such as directed evolution, may be used to improve enzymatic performance of key steps.

Reversal of the Natural Pathway for Acetate Conversion to Methane

One aspect of the present invention reverses the natural pathway converting acetate to methane, instead converting methane to acetate. Such reversal may be accomplished using a variety of approaches, for example by altering the activity or expression of key enzymes. Alteration can be achieved through manipulation and engineering techniques, for example by directed evolution, DNA shuffling, mutagenesis (e.g. saturation mutagenesis), enzyme redesign, heterologous expression of enzymes, and other molecular genetics approaches.

Key enzymes include those that carry out or contribute to the reactions set out in the above equations. For example, one or more of methylreductase (McrABC), coenzyme M (HS-CoM), bound methyltransferase complex (Mtr), soluble monomeric methyltransferase (CmtA), dehydrogenase/acetyl-CoA synthase (Cdh),the Rnf complex and methanophenazine (MP), phosphotransacetylase and acetate kinase, and methyl-CoM methylreductase may be specifically altered.

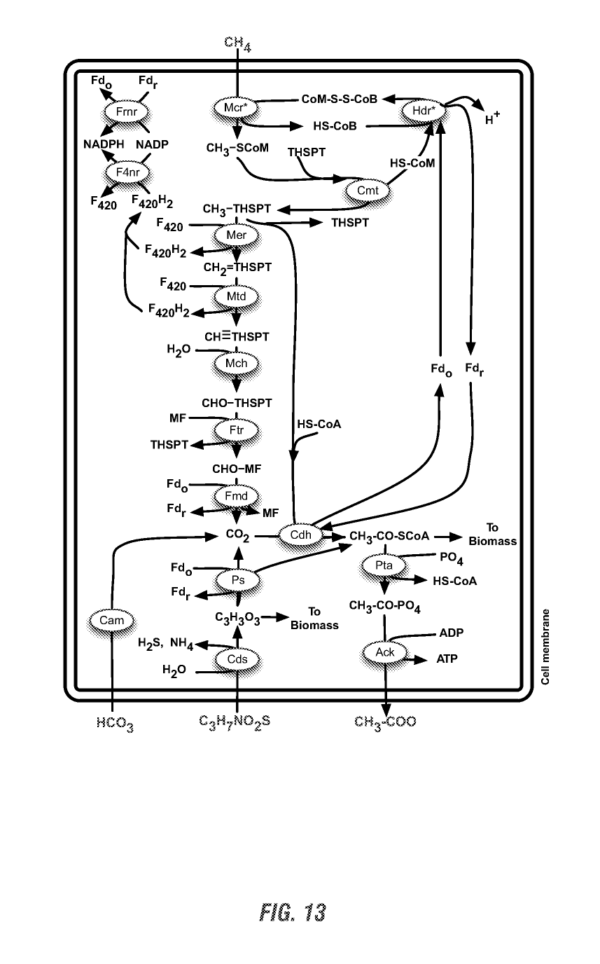

In one aspect, the natural pathway for acetate conversion to methane may be reversed along with modifications to specific aspects of the pathway. In an exemplary embodiment, shown in FIG. 13, modifications include reversal of the reaction catalyzed by methyl-coenzyme M methylreductase (Mcr), replacing membrane-bound complexes dependent on ion gradients, and diversion of acetyl-CoA to fuels and value-added products. In one more particular aspect, reversal of the reaction catalyzed by methyl-coenzyme M methylreductase (Mcr) may involve re-engineered (McrABC*) to oxidatively activate CH₄ and transfer the methyl group to coenzyme M (HS-CoM) at a significantly improved rate, for example, by overexpression through directed evolution. In another embodiment, Mcr may be engineered for improved activity, for example by DNA shuffling or, saturation mutagenesis to produce enzymes with enhanced, reversed Mcr activity.

In another aspect, replacing membrane-bound complexes may involve reversing or circumventing the natural sodium gradient, for example by replacing endogenous 8-subunit membrane-bound methyltransferase complex (Mtr) with a soluble monomeric methyltransferase (CmtA) that does not require a sodium gradient. In another aspect, a homolog of the CO dehydrogenase/acetyl-CoA synthase (Cdh) of Methanosarcina spp. may be provided or expressed, which is capable of consuming CO₂ and conserving electrons generated by oxidation of HS-CoB and HS-CoM to the heterodisulfide CoMS-SCoB with production of reduced ferredoxin (Fd).

In certain aspects, reversal of the natural pathway for acetate conversion to methane may involve altering expression or activity of enzymes that are common to all methanogenic pathways and are normally constitutively expressed. In other aspects, reversal of the pathway may involve expression of engineered enzymes under strong and/or inducible promoters. In other aspects, reversal of the pathway may involve utilizing enzymes that are naturally induced under certain conditions, for example growth in the presence of acetate. One or more of these approaches may be used in various combinations.

In another aspect, electricity may be used to supply reductant in place of CO. This approach may be preferred in light of reduction of carbon dioxide to methane by a methanogen using electricity to supply electrons.

DNA shuffling. Directed evolution or DNA shuffling is a powerful mutagenesis technique that mimics the natural molecular evolution of genes to efficiently re-design them. Its power lies in that it can introduce multiple mutations (many of them distal to the active site) into a gene to create new enzymatic activity (found by a suitable method of screening/selection). The power of the method stems from the fact that it can access improving mutations far away from the active site that are difficult to rationally predict using molecular modeling or intuition. Hence, it is not necessary to know the 3-D structure to optimize enzyme activity, and directed evolution identifies mutations that influence activity through subtle, long-range interactions. This method was developed by Willem Stemmer of Affymax Research Institute (now Maxygen) and consists of using PCR without oligo primers to re-assemble a gene from random 10-300 bp DNA fragments generated by first cleaving the gene with DNase. After re-assembling the original gene from these 300 bp fragments using a series of homologous recombinations and extensions with dNTPs and polymerase, normal PCR (with nested oligos) is performed using traditional oligos to yield the full-length gene with random mutations. The mutations arise from infidelity in the assembly process, PCR infidelity (polymerase base-reading errors), and errors introduced in the assembly process by insertion of mutated gene fragments (controlled by the researcher by adding specific oligos or DNA fragments from related but not identical genes). The advantages of this method are that DNA shuffling introduces mutations much more efficiently than other methods (e.g., unlike DNA shuffling, error-prone PCR and oligonucleotide cassette mutagenesis are not combinatorial), and it may be used to create a chimeric gene by reassembling closely-related genes (molecular breeding). This method has been used to increase β-lactamase antibiotic activity by 32,000-fold, to increase the fluorescence signal of the green fluorescent protein by 45-fold, and to evolve a fucosidase from β-galactosidase. Family shuffling has also been used to combine sequences of related enzymes to improve enzyme activity.

Saturation mutagenesis. Saturation mutagenesis is used to introduce all possible amino acids at key sites to explore a larger fraction of the protein sequence space than that of site-specific mutagenesis. It can provide much more comprehensive information than can be achieved by single-amino acid substitutions as well as overcome the drawbacks of random mutagenesis (e.g., error-prone PCR, DNA shuffling) in that a single mutation randomly placed in codons generates on average only 5.6 out of 19 possible substitutions (because it is very difficult to introduce more than one by change per codon using these methods and this is insufficient to introduce all amino acids at each codon). To determine the number of independent clones from saturation mutagenesis that need to be screened to ensure each possible codon has been tested, a multinomial distribution equation was developed; for example, it was determined that 292 colonies need to be screened if a single codon is changed.

These techniques (DNA shuffling and saturation mutagenesis) may be used to increase the effectiveness of Mcr in converting methane to methyl coenzyme M (CH₃—SCoM, step 1).

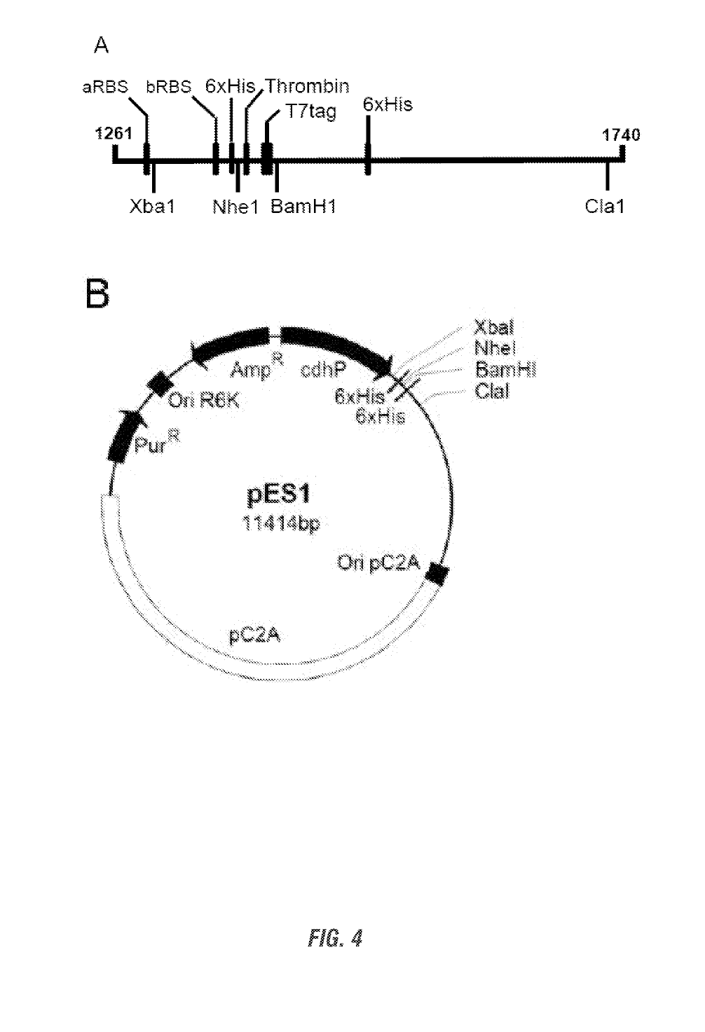

Molecular genetics with methanoarchaea. The pathways producing methane utilize several specialized metalloenzymes unique to the methanoarchaea that contain a diversity of metals and unique cofactors in the active site. The Inventors previously developed of a system for overproduction of recombinant metalloproteins in M. acetivorans to circumvent problems associated with overproduction in eukaryotic or bacterial systems. The system was validated by overproduction of catalytically active Cam, an iron-containing carbonic anhydrase that functions in the pathway (FIG. 4) for conversion of acetate to methane. The system is based on a plasmid shuttle vector with selectable markers for both M. acetivorans and E. coli (FIG. 4). The promoter for the cdh operon encoding CODH/ACS is ligated upstream of a multiple cloning site. In vivo studies using translational fusions with LacZ show this promoter to have 40 to 60-fold greater expression than basal levels when M. acetivorans is grown on acetate. The expression vector has the same plasmid backbone as pEA103, which is present at low copy numbers of approximately 15 copies per cell during growth of M. acetivorans on acetate and is stable for at least 22 generations without selection negating the requirement for puromycin during high volume scale-up. This construct includes a multiple cloning site flanked by sequence for 6× His translational fusions to allow for optional C- and N-terminal fusion proteins.

Rapid generation of metabolic models. Genome-scale models have been developed for a wide variety of microorganisms including pathogens, archaea and plants. Table 1 summarizes the reconstruction efforts and highlights size statistics and quality metrics. Specificity, as shown in Table 1, is defined as the % correctly-identified true positives such as essential genes or feasible growth substrates while sensitivity is the % of correctly-identified true negatives such as non-essential genes or substrates that do allow for growth. Systematic procedures have been used to refine and improve the prediction accuracy of the draft reconstructed metabolic models. Examples include model refinement via GapFind and GapFill to unblock biomass precursors and reconnect unreachable metabolites, as well as evaluation and improvement of model performance when compared to in vivo gene essentiality or synthetic lethality data (if available) using the GrowMatch and Extended GrowMatch procedures. A Knowledgebase called MetRxn has also been developed for the reaction/metabolite data standardization, correction, and congruency to aid model reconstruction efforts.

TABLE 1
Reconstructed models, size statistics and quality metrics.

| | Model | | | | | |
|---|---|---|---|---|---|---|
| Organism | Name | Genes | Metabolites | Reactions | Specificity | Sensitivity |
| Mycoplasma | iPS189 | 189 | 274 | 262 | 87% | 89% |
| genitalium | | | | | | |
| Salmonella | iMA945 | 945 | 1,036 | 1,964 | 67% | 92% |
| Typhimurium | | | | | | |
| LT2 | | | | | | |
| Methanosarcina | iVS941 | 941 | 708 | 705 | 90% | 84% |
| acetivorans | | | | | | |
| Zea mays | iRS1563 | 1,563 | 1,825 | 1,985 | N/A | N/A |
| Cyanothece sp. | iCyt773 | 773 | 811 | 946 | N/A | N/A |
| ATCC 51142 | | | | | | |
| Synechocystis | iSyn731 | 731 | 996 | 1,156 | N/A | N/A |
| sp. PCC 6803 | | | | | | |

A genome-scale metabolic model has been developed for Methanosarcina acetivorance (iVS941). M. acetivorans, with a genome size of ˜5.7 mb, is the largest sequenced archaeon methanogen and unique amongst the methanogens in its biochemical characteristics. The generated model iVS941 accounts for 941 genes, 705 reactions and 708 metabolites. Using a systematic procedure relying on the GapFind, GapFill and the GrowMatch procedures enabled sequential evaluation and improvement of model capabilities. The completed model has metabolites that can be produced (87%) and it has a high agreement of 93.3% against published in vivo growth data across different substrates and genetic perturbations with specificity of 81% and sensitivity of 89.7%. The model also correctly recapitulates metabolic pathway usage patterns of M. acetivorans such as the indispensability of flux through methanogenesis for growth on acetate and methanol and the unique biochemical characteristics under growth on carbon monoxide.

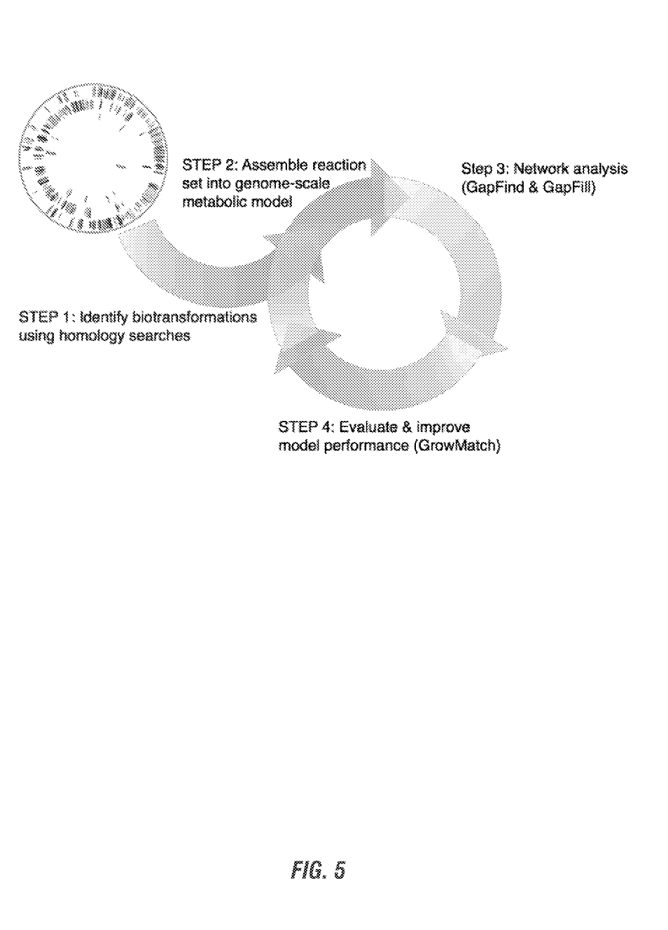

Metabolic model reconstruction pipeline. The metabolic model reconstruction process follows four major steps (see FIG. 5): (1) Reconstruction of draft model via automated homology searches for the identification of native biotransformations or by using the available online tools such as the Model Seed; (2) Generation of a computations-ready model after defining biomass equation and system boundary and establishing gene-protein-reaction (GPR) associations; (3) Model refinement via GapFind and GapFill; (4) Further evaluation and improvement of model performance when compared to in vivo gene essentiality or synthetic lethality data (if available) using the GrowMatch and/or Extended GrowMatch procedures. Model reconstruction is followed by minimal defined medium component elucidation.

The model reconstruction and refinement tools may be used in conjunction with the ModelSEED resource and MetRxn knowledgebase to rapidly reconstruct highly curated metabolic models for methanogens. The energetics, electron flow and regulation of export/import of various metabolites and signaling molecules may be described.

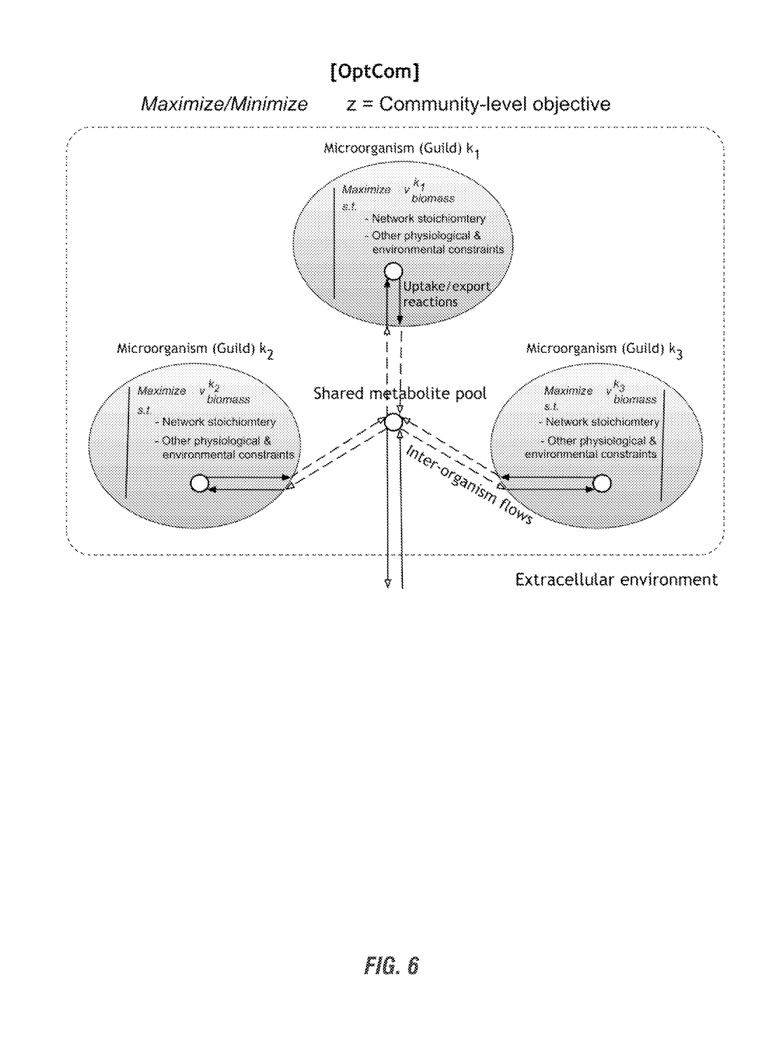

Community metabolic modeling using multi-level and multi-criteria optimization techniques. OptCom is a flux balance analysis framework for microbial communities using genome-scale metabolic models, which relies on a multi-level and multi-objective optimization formulation to properly describe trade-offs between individual vs. community level fitness criteria (see FIG. 6). In contrast to earlier approaches that rely on a single objective function, OptCom considers species-level fitness criteria for the inner problem while relying on community-level objective maximization for the outer problem. OptCom is general enough to capture any type of interactions (positive, negative or combinations thereof) and is capable of accommodating any number of microbial species (or guilds) involved.

An automated computational workflow may be used to select, from an ensemble of microbial models, what member would lead to the most efficient utilization of available metabolic resources. Such a workflow has been used in the design of the synthetic microbial community composed of M. acetivorans co-cultured with an acetate-utilizing species to alleviate the thermodynamic unfavorable conditions.

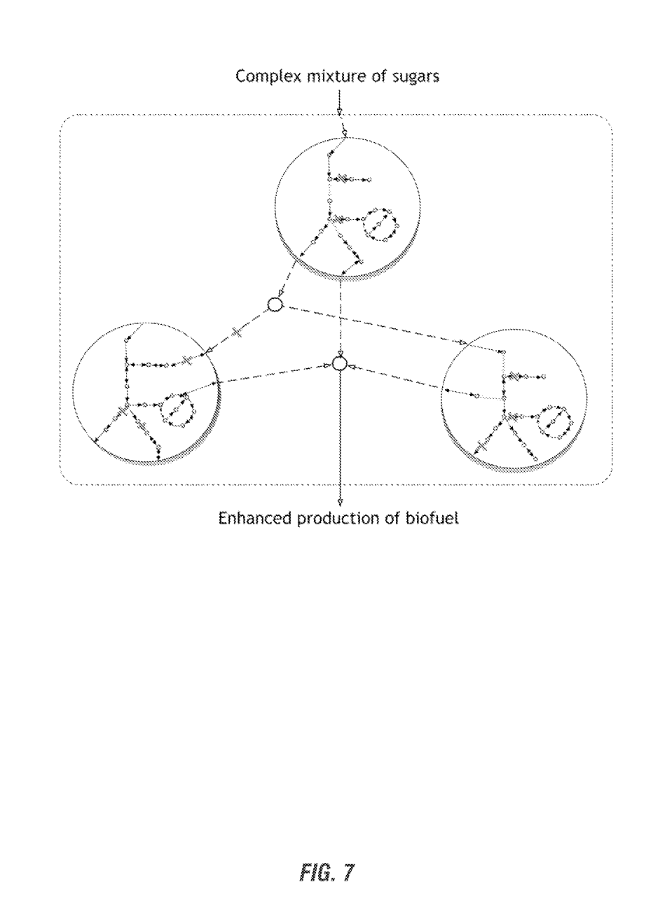

In addition, targeted gene knockouts may be introduced. These genetic manipulations may be warranted for forcing a desired obligatory syntrophic relation in the community (so as metabolic flows can be uniquely attributed) or as part of optimizing the production of a targeted product metabolite. To this end, computational strain design procedures such as OptKnock and OptForce may be extended to a multi-species setting (see FIG. 7).

De novo enzyme design. Computational protein design techniques may be used in support of the enzyme engineering for a targeted pathway.

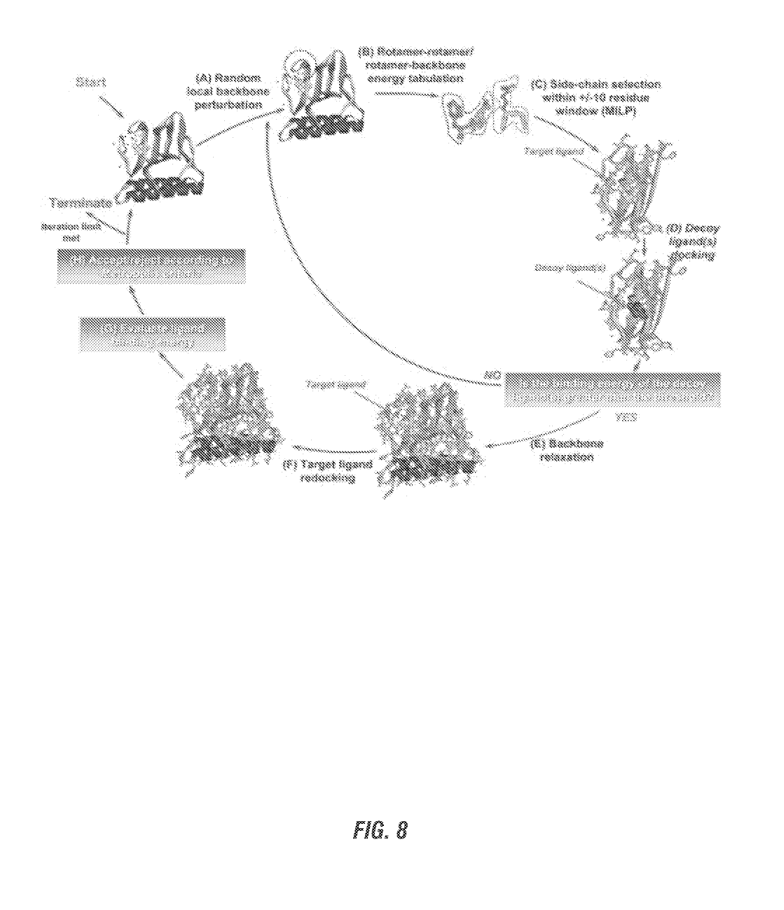

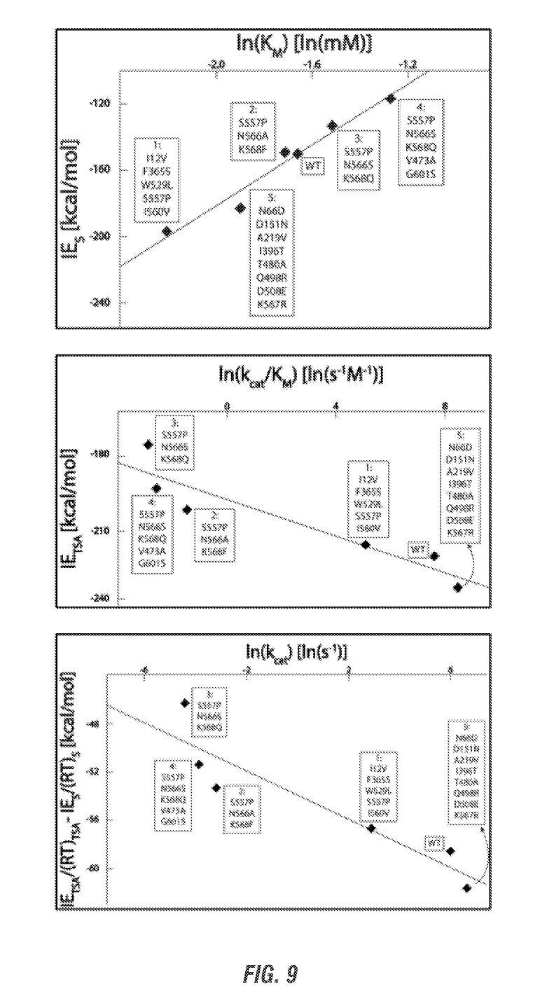

In FIG. 8 we show the workflow for Iterative Protein Redesign and Optimization (IPRO). First, a local region of the protein (1-5 consecutive residues around the targeted ligand) is randomly chosen for perturbation. The φ and ψ angles of the targeted position are perturbed by up to 5°. Next, all amino acid rotamers consistent with these torsion angles are selected at each position from the Dunbrack and Cohen rotamer library. Rotamer-backbone and rotamer-rotamer energies are calculated for all of the selected rotamers. This is followed by a binding energy minimization using a mixed-integer linear programing (MILP) formulation to select the optim al rotamer at each of these positions. Once an optimal arrangement of rotamers has been selected, a CHARMM energy minimization is carried out to adjust to the changes in the new side chains. Finally, the ligand is redocked with the protein and the protein-ligand energy is computed using CHARMM energy functions. A redesign step is accepted if the energy is the best identified thus far, otherwise the Metropolis criterion is used to decide whether to accept or reject the step. In addition to considering a single ligand, IPRO can consider multiple objectives simultaneously (e.g. suppressing binding to decoy ligands, creating favorable binding to multiple ligands, etc.). By iterating through backbone perturbations and optimal rotamer selections, IPRO can successfully identify mutations that lead to improved protein properties. It has been successfully applied to alter the specificity of Candida boidinii xylose reductase from NADPH to NADH, to transfer a calcium-binding pocket from thermitase into the first domain of CD2, and to design antibody complementarity determining regions.

Here, an enzyme design method, termed OptZyme may be used. The initial step of OptZyme is assessing whether the enzyme should be redesigned to modify k_{cat}, K_{M}, k_{cat}/K_{M}, or a combination of these kinetic properties. If the objective kinetic property is either k_{cat} or k_{cat}/K_{M} an appropriate transition state analogue (TSA) must be chosen that resemble the transition state of the desired reaction. TSA selection can be guided from the transition state structure, resolved from quantum mechanics calculations, or reported inhibitory compounds. Next, key catalytic contacts are identified, and harmonic restraints are imposed to preserve enzymatic activity during the redesign process. Finally, IPRO is employed to find mutations that minimize interaction energy with the appropriate substrate. Multiple IPRO trajectories can be used to identify various mutants with low objective function values. These values can be further validated by modeling the enzyme's active site with quantum mechanics, while representing the remainder of the enzyme with molecular mechanics.

For the E. coli β-glucuronidase (GUS), K_{M} correlates (R²=0.960) with the interaction energy with the native substrate analogue, while k_{cat}/K_{M} correlates (R²=0.864) with the transition state analogue (TSA) D-glucaro-1,5-lactone (see FIG. 9). Also, k_{cat} correlates (R²=0.854) with a weighted difference of interaction energies between the TSA and native substrate analogue. The correlating expressions were derived using the Eyring-Polyani equation and the relationship between Gibb's free energy and equilibrium constants. The implicit assumptions within these expressions include that the equilibrium constant of product release must lie far to the right, stabilization of the TSA corresponds to stabilization of the transition state, and conformational rearrangements do not occur upon substrate binding. OptZyme is unique as it provides a theoretical framework for making use of TSA binding calculations to inform enzyme design. OptZyme offers advantages over existing enzyme design protocols because it does not require full convergence of quantum mechanics calculations but instead incorporates information from preliminary quantum mechanics calculations. It may be used to design focused combinatorial libraries to improve enzyme activity and shed light onto structural differences that underpin improved K_{M}, k_{cat}, or k_{cat}/K_{M}.

OptZyme may be used to alter enzyme specificity from a native substrate analogue, para-nitrophenyl-β, D-glucuronide, to a novel substrate, para-nitrophenyl-β, D-galactoside. OptZyme was used to improve k_{cat}, K_{M}, and k_{cat}/K_{M} for both substrates. Differences were observed between the corresponding libraries for both substrates, exhibiting the power of OptZyme to distinguish between similar substrates. In addition, separate OptZyme runs may be used to decipher between essential residues for substrate binding and important residues for substrate turnover. Using various objectives within OptZyme, we were able to discover that large, polar side chains (e.g. lysine and aspartate) favored the improvement of k_{cat}/K_{M} for GUS. This tendency was attributed to the more flattened geometry of the TSA relative to the substrate. Furthermore, this propensity could be used to bias combinatorial libraries with large, polar amino acids if the goal of the redesign is improving k_{cat}/K_{M}. This computational infrastructure may be deployed and fine-tuned for enzymes whose activity is identified as limiting in our assembled pathway.

Novel biocatalysts through DNA shuffling and saturation mutagenesis. DNA shuffling has been successfully deployed by for monooxygenases, dioxygenases, epoxide hydrolases, and biofilm regulators. Hence, such approaches have been used to create better catalysts as well as to create better regulators for synthetic circuits.

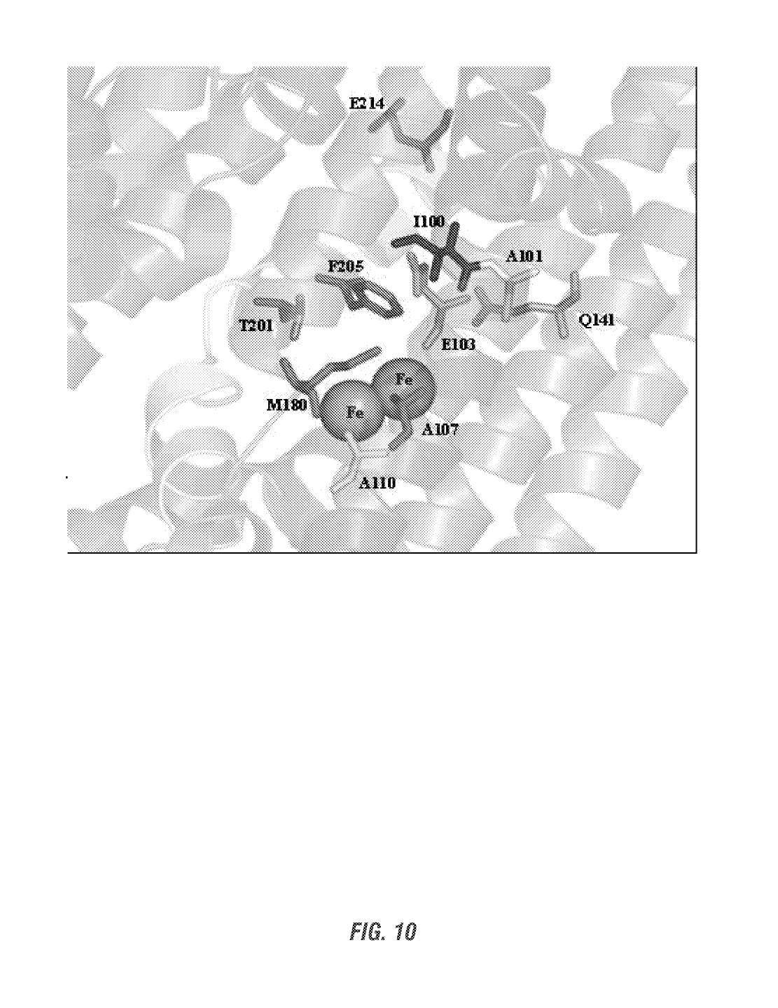

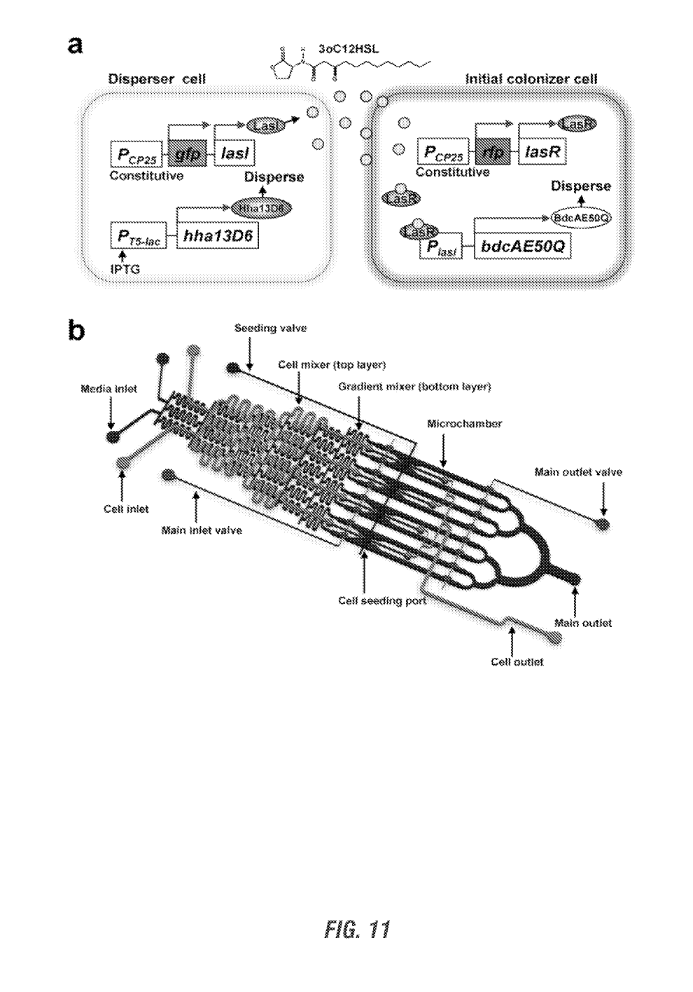

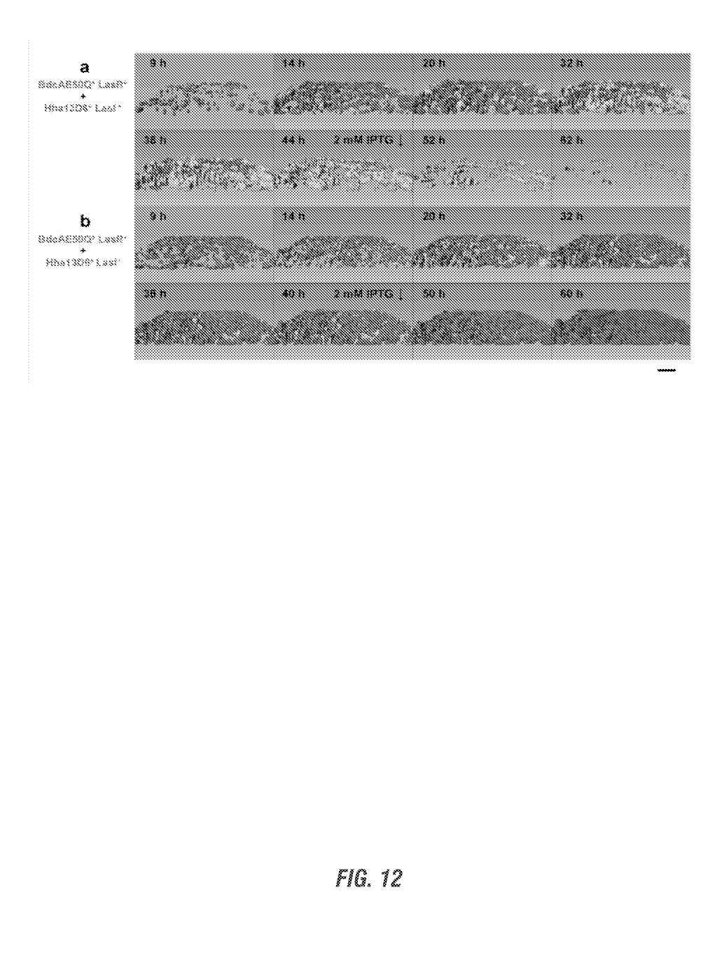

Soluble methane monooxygenase (sMMO) was first cloned with activity in 1994 in a heterologous host. Since then the related toluene monooxygenases in E. coli have been evolved for various applications including the production of 8 industrial compounds that could not previously be made by bacteria or enzymes (nitrohydroquinone, 4-methylresorcinol, 1-hydroxyfluorene, 3-hydroxyfluorene, 4-hydroxyfluorene, 2-naphthol, 2,6-dihydroxynaphthalene, and 3,6-dihydroxyfluorene) and have discovered six residues that influence catalysis (gate residue I100, A101, A107, A110, M180, and gate residue E214, FIG. 10). Previous work by the Inventors has also determined the wild-type toluene monooxygenases are capable of three successive hydroxylations of benzene and determined the physiological relevance of these reactions; found that protein engineering may be used to create the first meta-hydroxylating enzyme for toluene so that regioselective hydrolysis may be controlled for the first time; shown that protein engineering may be used to control the regiospecific oxidation of nitroaromatics, methylaromatics, and methoxyaromatics; and demonstrated that protein engineering may be used to make a rainbow of indigoid compounds for dyes and pharmaceuticals.

The Inventors have also used these methods to evolve dioxygenases for green chemistry applications and for bioremediation. For example, the Inventors evolved the first two enzymes in the pathway for 2,4-dinitrotoluene (2,4-DNT) degradation, and the evolution of the large subunit of 2,4-DNT dioxygenase led to an increase in the rates of degradation of 2,3-DNT, 2,4-DNT, 2,5-DNT, 2,6-DNT, 2-NT, and 4-NT and to the first enzyme capable of degrading 2,3-DNT and 2,5-DNT; the evolution of 4-methyl-5-nitrocatechol monooxygenase (the second enzyme in the degradation of 2,4-DNT) led to a broadening of the enzyme substrate range to include 4-nitrophenol and 3-methyl-4-nitrophenol. The production of engineered enzymes by the Inventors has been described previously.

Synthetic circuits, and engineered process regulators. In previous work determining that indole decreases biofilm formation, the Inventors also developed the first synthetic signaling circuit to control biofilm formation and used it to control the biofilm formation of E. coli and Pseudomonas fluorescens by manipulating the extracellular concentration of the signal indole. Based on this previous work, protein engineering may be used to create cell novel catalysts as well as circuits for various applications, including engineered CH₄-to acetate pathways.

Engineering Promoters

In certain embodiments, expression of one or more components of the engineered pathway for conversion of methane to acetate may be under the control of a particular promoter, for example an inducible promoter. In an exemplary embodiment expression of one or more components may be under the control of a tetracycline-dependent, for example, the promoter driving expression of one or more genes encoding a component of the engineered pathway may include additional nucleotide sequences permitting tetracycline inducibility.

### Deposit of Biological Material

The following biological material has been maintained by Applicant since prior to the filing date of this application. Access to biological material will be available during the pendency of the application to the Commissioner of Patents and Trademarks and persons determined by the Commissioner to be entitled thereto upon request.

| | | Accession | |
|---|---|---|---|
| Strain | Depository | number | Date of deposit |
| Methanosarcina acetivorans | | | |

### Strain C2A/pES1-MATmcr

The biological material listed above will be deposited under the terms of the Budapest Treaty on the International Recognition of the Deposit of Microorganisms for the Purposes of Patent Procedure. The listed deposit will be maintained in the indicated international depository for at least 30 years and will be made available to the public upon the grant of a patent disclosing it. All restrictions imposed by the depositor on the availability to the public of the deposited materials will be irrevocably removed upon the granting of a patent in this application. The availability of a deposit does not constitute a license to practice the subject invention in derogation of patent rights granted by government action.

The examples below are provided only for illustrative purposes and not to limit the scope of the present invention. Numerous embodiments within the scope of the claims will be apparent to those of ordinary skill in the art, thus the following non-limiting examples only describe particular embodiments of the invention. The present invention relates to colorimetric read-out systems capable of detecting a variety of biomolecules, including methods and kits relating thereto.

To facilitate a better understanding of the present invention, the following examples of preferred or representative embodiments are given. In no way should the following examples be read to limit, or to define, the scope of the invention.

### EXAMPLES

Engineered Reversal of the Natural Pathway for Acetate Conversion to Methane

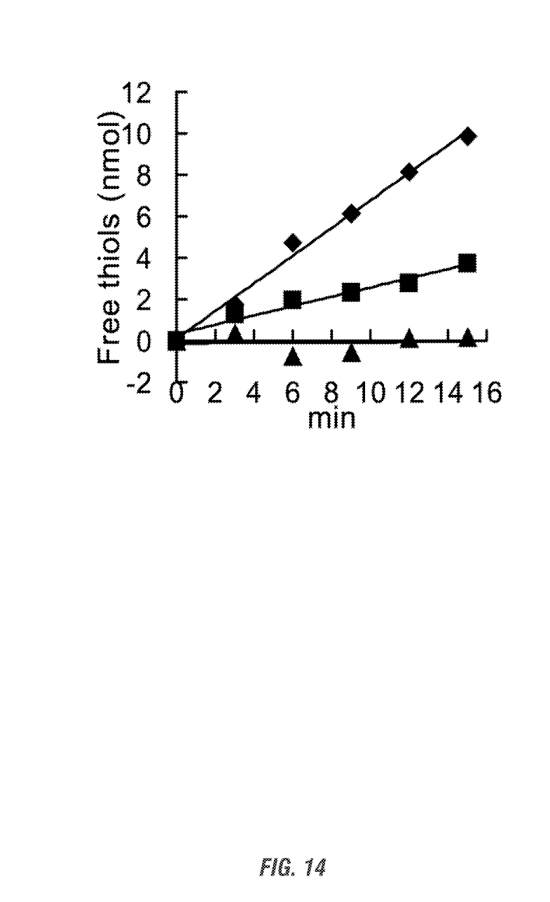

An exemplary engineered CH₄-to-acetate pathway (Phase I) is shown in FIG. 13, which includes a reversal of the natural pathway with key modifications. Step 1 is catalyzed by methylreductase (McrABC) re-engineered (McrABC*) to oxidatively activate CH₄ and transfer the methyl group to coenzyme M (HS-CoM) at a significantly improved rate. We have documented the ability of M. acetivorans to oxidize CH₄ that almost certainly is catalyzed by the McrABC. Indeed, the McrABC of a distantly related methanogen is reported to catalyze the reverse reaction⁸⁹. Reverse activity of the M. acetivorans McrABC may be confirmed by asking if the carbon of ¹³C-labeled CH₄ appears in the methyl group of CH₃—SCoM. Directed evolution may be applied to the enzyme to enhance the rate of the reaction by taking advantage of our demonstrated ability to over-produce metalloenzymes in M. acetivorans. Significant rates of methane conversion to acetate may be achieved since CH₃—SCoM is removed as a product of McrABC (Steps 2-5). The reverse of Step 2 is catalyzed in the acetate-to-CH₄ pathway by an 8-subunit membrane-bound methyltransferase complex (Mtr) that generates a sodium gradient driven by the exergonic reaction. Catalysis by Mtr of endergonic Step 2 (FIG. 13) requires a sodium gradient which is absent in the exemplary engineered pathway. A soluble monomeric methyltransferase (CmtA) in M. acetivorans, not requiring a sodium gradient, may be used to replace Mtr in the engineered pathway. Step 3 is catalyzed by a homolog of the CO dehydrogenase/acetyl-CoA synthase (Cdh) of Methanosarcina spp. that we have shown is reversible consuming CO₂ and conserving electrons generated by oxidation of HS-CoB and HS-CoM to the heterodisulfide CoMS-SCoB (Step 4) with production of reduced ferredoxin (Fd) re-oxidized in Step 3. A soluble ferredoxin:heterodisulfide oxidoreductase system (Step 4) is present in acetate-grown M. acetivorans (FIG. 14) that bypasses the membrane-bound electron transport chain of the acetate-to-CH₄ pathway involving the Rnf complex and methanophenazine (MP) greatly simplifying the proposed engineered pathway. Acetate production in M. acetivorans is catalyzed by phosphotransacetylase and acetate kinase of Methanosarcina spp. catalyzing Step 5 that yields one ATP required for growth and maintenance. Enzymes catalyzing Steps 1 and 4 are common to all methanogenic pathways and constitutively expressed. The mcrABC* gene encoding the engineered methyl-CoM methylreductase and cmtA encoding the soluble methyltransferase, catalyzing reactions 1 and 2, will be placed behind the strong mcrB promoter using methods we have described. Where necessary, promoters may be engineered with additional sequences for tetracycline-dependent expression using methods we have described. Enzymes catalyzing Steps 3 and 5 are naturally induced in the presence of acetate, a product of the engineered pathway, and therefore expected to be induced to high levels in the engineered pathway. Only one ATP is produced in the reverse pathway ensuring that the great majority of acetate produced will be excreted for conversion to liquid fuels via Phase II and not diverted into biomass for growth. The engineered strain may be cultured with acetate to ensure high level expression of the requisite enzymes. If synthesis of the engineered McrABC* and CmtA inhibit growth with acetate, strains may be developed with tetracycline-dependent promoters and the genes induced at the end of growth. As the CH₄-to-acetate pathway is thermodynamically unfavorable (ΔG°=+36 kJ), the engineered M. acetivorans strain will be co-cultured with an acetate-utilizing (acetate+SO₄⁻²=2HCO₃⁻+HS⁻ ΔG°=−71.7 kJ) sulfate-reducing species in experiments documenting and characterizing the engineered pathway in M. acetivorans. Although not reconstituted with purified species, anaerobic oxidation of methane coupled to sulfate reduction is well documented in the environment⁹¹ validating the proposed co-culture approach. Although not a viable route to liquid fuel production, the co-culture may provide a way of testing the feasibility of reversing the methanogenesis pathway for application in phase II.

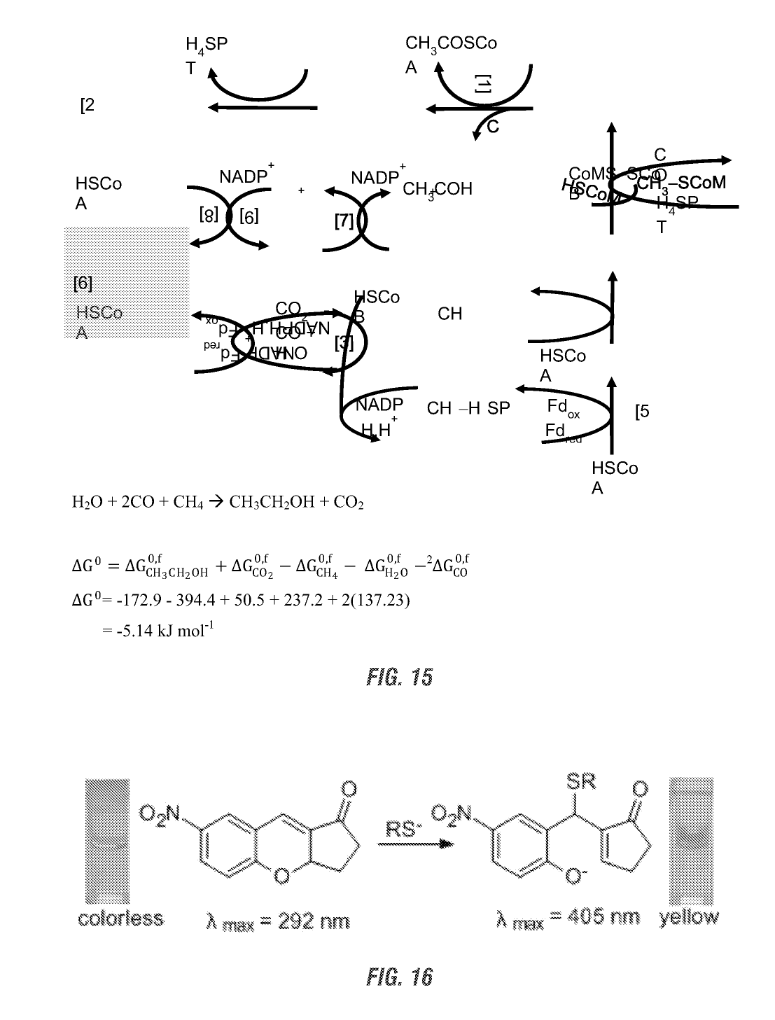

CO-assisted conversion of methane to liquid fuels. The engineered pathway shown in FIG. 13 may be built upon to include a engineered pathway converting methane to liquid fuels thermodynamically assisted with the reductant CO. FIG. 15 shows an example of a pathway leading from methane to ethanol. Note that the pathway is modular in nature and can be modified for producing other liquid fuels. The pathway shown in FIG. 15 is further engineered by introducing three genes into the M. acetivorans genome encoding a ferredoxin:NADP oxidoreductase (Fnr) and NADP⁺-dependent alcohol and aldehyde dehydrogenases (Adh and Ald). The fnr gene is obtained from plant, Escherichia coli⁶ or cyanobacteria, and the adh and ald genes from Moorella sp. HUC22-1. Although not yet purified and characterized to validate activity, the genome of M. acetivorans contains genes (MA4079 and MA1902) annotated as NADP⁺-dependent aldehyde and alcohol dehydrogenases that could be used in place of the Moorella genes if activity is validated by heterologous production in E. coli and characterization. The genes will be placed behind the strong mcrB promoter from M. acetivorans and introduced into the genome using methods we previously described. The promoter for the Fnr gene will also be engineered for tetracycline-dependent expression as we have previously described. The engineered strain may be cultured with acetate to high density followed by addition of tetracycline to induce fnr.

TABLE 2
Reactions and associated ΔG°′ values (standard conditions) for the proposed
methane-to-ethanol pathway.

| | | ΔG°′ |
|---|---|---|
| Step | Reaction | (kJ mol⁻¹) |
| 1 | CH₄ + CoM—S—S—CoB → | +45.0 |
| | H—S—CoB + CH₃—S—CoM | |
| 2 | CH₃—S—CoM + H₄SPT → | +30.0 |
| | CH₃—H₄SPT + H—S—CoM | |
| 3-4 | H—S—CoB + H—S—CoM + NADP⁺ → | +34.9 |
| | CoM—S—S—CoB + NADPH,H⁺ | |
| 5 | CH₃—H₄SPT + CO + CoA—SH→ | −65.0 |
| | CH₃COSCoA + H₄SPT + H₂O | |
| 6-7 | H₂O +CO + NADP⁺ → | −42.1 |
| | CO₂ + NADPH,H⁺ | |
| 8-9 | CH₃COSCoA +2NADPH,H⁺ → | −8.2 |
| | CH₃CH₂OH + 2NADP⁺ + CoA—SH | |
| Sum | H₂O + 2CO + CH₄ → | −5.3 |
| | CH₃CH₂OH + CO₂ | |

Table 2 lists the engineered pathway reaction steps and associated ΔG°′ for each under standard conditions. The pathway initiates with oxidation of CH₄ (step 1) and transfer of the methyl group to form CH₃—H₄SPT (step 2) catalyzed by McrABC* and CmtA. The HS-CoM and HS-CoB produced in steps 1 and 2 are oxidized by the soluble ferredoxin:heterodisulfide oxidoreductase system expressed during growth with acetate to regenerate CoMS-SCoB (step 3). Oxidized ferredoxin is regenerated by transfer of electrons to NADP⁺ catalyzed by Fnr (step 4). CO supplies the carbonyl group for coupling with the methyl group of CH₃—H₄SPT producing acetyl-CoA (step 5) catalyzed by the Cdh synthesized during growth with acetate¹¹². A second molecule of CO is oxidized by the multi-functional Cdh reducing ferredoxin (step 6) that is re-oxidized by Fnr (steps 6-7) contributing to the NADPH,H⁺ pool. The two molecules of NADPH and H⁺ are re-oxidized by Ald and Adh in steps 8-9 producing ethanol.

The acetate-grown and tetracycline-induced engineered M. acetivorans strain may be transferred to a buffered resting cell solution under one atmosphere containing one-third CH₄ and two-thirds CO. Factors (temperature, pH, gas composition and partial pressures, and ethanol concentration) determining the rate, product composition, stoichiometry and ethanol tolerance will be examined. High tolerance to CO partial pressures (at least up to three atmospheres) are possible based on published accounts for growth on CO.^{58, 78} Although acetate kinase and phosphotransacetylase will be present (catalyzing acetyl-CoA+ADP→ATP+acetate) in the acetate-grown cells, little acetate production is anticipated based on unfavorable thermodynamics.

Electricity may be used to supply reductant in place of CO. This approach is encouraged by the published account for reduction of carbon dioxide to methane by a methanogen using electricity to supply electrons.

Calculating the ΔG of reaction under non-standard conditions for methane to ethanol. The non-standard ΔG (in kJ mol⁻¹) is calculated by the following relation:

ΔG=ΔG′⁰+RT.ln(Q) [Eq. 11]

where, ΔG′⁰ is the standard Gibbs Free Energy change of reaction (in kJ mol⁻¹), R is the Universal Gas constant (8.314×10⁻³ kJ mol⁻¹ K⁻¹), T is temperature (298 K), and Q is the reaction quotient.

Under standard conditions of 1 atm and equimolar composition of CO, CH₄ and CO₂ in gaseous phase, the non-standard ΔG of the reaction is calculated in Table 3 for the CO-assisted phase II reaction:

H₂O+2CO+CH₄→CH₃CH₂OH+CO₂ [Eq. 12]

TABLE 3
Calculation of the non-standard ΔG of the net reaction at
atmospheric pressure. (Eq. 11) for (Eq. 12)

| | Henry's law | Partial | Aqueous | |
|---|---|---|---|---|
| | constant | Pressure | concentration | Activity |
| Metabolites | K_{H} (atm/M) | Pᵢ (atm) | (cᵢ [M]) | (γ) |
| CH₄ | 716.44 | 1/3 | 4.65e−4^{a} | 4.65e−4 |
| CO | 1052.63 | 1/3 | 3.1667e−4^{a} | 3.167e−4 |
| H₂O | — | — | 50^{b} | 0.9009 |
| CH₃CH₂OH | — | — | 0.22^{c} | 0.22 |
| CO₂ | 28.66 | 1/3 | 0.0116^{a} | 0.0116 |

^{a}Using Henry's Law: Pᵢ = K_{H} · cᵢ
^{b}Concentration of water inside the cell is slightly less than pure water (55.5M)
^{c}Assumed maximum ethanol tolerance 10 g/l → 0.217M
Q = (γ_{Ethanol} × γ_{CO}_{₂})/(γ_{H}_{₂}_{O} × γ_{CH}_{₄} × γ_{CO}^{²}) = 6.0798e+07
ΔG = −5.2 + 8.314 * 10 − 3 * 298 * ln(0.73814), +39.17 kJ mol⁻¹

As shown in Table 3, the net reaction is thermodynamically unfavorable (i.e., ΔG=+39.17 kJ mol⁻¹). It is contemplated that this unfavorable condition can be alleviated by increasing the pressure to 20 atm in order to enhance the solubility (and hence activity) of CH₄ and CO. Assuming 1 mM aqueous concentration of ethanol and CO₂, this analysis revealed that the optimal partial pressures of CO and CH₄ minimizing the ΔG of the reaction and reducing it to a negative value are 13 and 7 atm, respectively (see Table 4). Therefore, at sufficiently high pressures, with a 2:1 ratio of CH₄ and CO, it is possible to ensure thermodynamic feasibility of the reaction (i.e., ΔG=−0.76 kJ mol⁻¹).

TABLE 4
Calculation of the non-standard ΔG of the net reaction at 20
atm pressure. [Eq. 11] for [Eq. 12].

| | Henry's law | Partial | Aqueous | |
|---|---|---|---|---|
| | constant | Pressure | concentration | Activity |
| Metabolites | K_{H} (atm/M) | Pᵢ (atm) | (cᵢ [M]) | (γ) |
| CH₄ | 716.44 | 7 | 0.01235^{a} | 0.01235 |
| CO | 1052.63 | 13 | 0.00977^{a} | 0.00977 |
| H₂O | — | — | 50^{b} | 0.9009 |
| CH₃CH₂OH | — | — | 0.001 | 0.001 |
| CO₂ | 28.66 | 0.02866 | 0.001 | 0.001 |

^{a}Using Henry's Law: Pᵢ = K_{H} · cᵢ
^{b}Concentration of water inside the cell is slightly less than pure water (55.5M)
Q = (γ_{Ethanol} × γ_{CO}_{₂})/(γ_{H}_{₂}_{O} × γ_{CH}_{₄} × γ_{CO}^{²}) = 0.73814
ΔG −5.2 + 8.314 * 10 − 3 * 298 * ln(0.73814), = −0.75738 kJ mol⁻¹

Protein & pathway engineering. In addition to the assembly of the pathway, improvements in the catalytic efficiency of Steps 1 through 5 (FIG. 13) are contemplated. For example, Mcr converting methane to methyl coenzyme M (CH₃—SCoM) (under Step 1) may be engineered for improved activity. DNA shuffling may be performed using the procedure of Stemmer modified by Zhao and Arnold. In addition, saturation mutagenesis may be used to introduce all possible amino acids at key sites to explore a larger fraction of the protein sequence space⁸⁵.

The screen for enhanced, reversed Mcr activity may be conducted in M. acetivorans due to the need for the nickel cofactor, hydroporphinoid nickel complex coenzyme F₄₃₀, that is not synthesized in Escherichia coli. The E. coli-M. acetivorans shuttle vector pWM321 may be used to express Mcr from M. acetivorans using the constitutive M. acetivorans promoter P_{tbp}. To gauge higher Mcr activity (Step 1, FIG. 1), a colorimetric assay may be used based on detecting production of HS-CoB (product of the methane to CH₃—SCoM reaction) which has a reactive, terminal thiol group; the thiol of HSCoB will be detected by using a chromene derivative, 7-nitro-2,3-dihydro-1H-cyclopenta[b]chromen-1-one (7NDCC), which has high selectivity and sensitivity for detecting thiol species such as those in cysteine and glutathione in aqueous solution. Upon addition of 7NDCC to thiols, a rapid yellow color change occurs (see FIG. 16). This is a novel approach that allows for utilizing plates having a particular number of wells (e.g. 96) to rapidly screen thousands of potential protein variants.

After transforming M. acetivorans via a liposome-mediated protocol, to conduct the enzymatic screen in a 96-well format, an anaerobic chamber may be used to keep Mcr active as well as to provide the proper concentration of methane to drive the reaction. M. acetivorans cells overexpressing the Mcr variants formed from DNA shuffling will be screened spectrophotometrically for HSCoB production in cell lysates by detecting the yellow color formed with 7NDCC. Cells containing Mtr variants may be grown in 300 μL of high-salt medium with shaking in Costar 96-well plates (Corning, Corning, N.Y.). The cells may be harvested at mid-log phase by filtering 200 μL of the cell cultures using MultiScreen-GV 96-well filter plates (Millipore, Bedford, Mass.). The collected cells may then be washed with 200 μL 50 mM Tris-HCl, pH 7.4, suspended in 200 μL of 10 mM HEPES buffer, and sonicated using a 96-well plate sonicator (MISONIX Sonicator 4000). The cell lysates may be exposed to methane (0.5 atm) and heterodisulfide (1 mM) then 7NDCC (25 μM) may be added, and the appearance of HSCoB may be quantified spectrophotometrically at 405 nm using a 96-well plate reader (Multiscan RC, Labsystems, Helsinki, Finland). Lysates from wells with the greatest absorbance (largest amount of HSCoB) may be saved, re-checked in additional 96-well plates, and the plasmids isolated from the highest-expressing strains may be used for subsequent rounds of shuffling, saturation mutagenesis, and DNA sequencing. In this way, Mcr may be engineered to improve dramatically the rate limiting step (Step 1) in FIG. 13, conversion of methane into CH₃—SCoM. To determine the number of independent clones from saturation mutagenesis that need to be screened to ensure each possible codon has been tested, a multinomial distribution equation may be used. The recent structure obtained for the Mcr from Black Sea mats may be used to determine residues for mutagenesis to increase reaction rates.

De novo enzyme design. Along with the protein engineering using combinatorial screening (both directed evolution and site directed mutagenesis), de novo enzyme design computational tools may be used. An enzyme design method such as OptZyme may be used. OptZyme implements as surrogates for the typically unknown transition state (TS) structure, transition state analogue (TSA) compounds which are known for many enzymatic reactions. Metrics were derived that correlate enzyme-TSA interaction energies with enzyme activity (i.e., k_{cat} and k_{cat}/K_{M}). By identifying mutations that minimize the interaction energy of the enzyme with its TSA rather than its native substrate, transition state energy barrier lowering is presumably achieved. It is contemplated that integrated use of computations and combinatorial screening will help lead to improved enzymatic performance in the pathway. In addition, the M. acetivorans metabolic model iVS941 (705 reactions and 708 metabolites) may be used as a blue print for exploring the impact of any proposed pathway construct on system-wide metabolism including coupling of any proposed pathway with metabolic conversions already present in M. acetivorans (or in co-culture with other organisms) with a negative free energy to drive the pathway in the intended direction.

Reversal of Methanogenic Pathways and Extra-Cellular Electron Donation by Mcr.

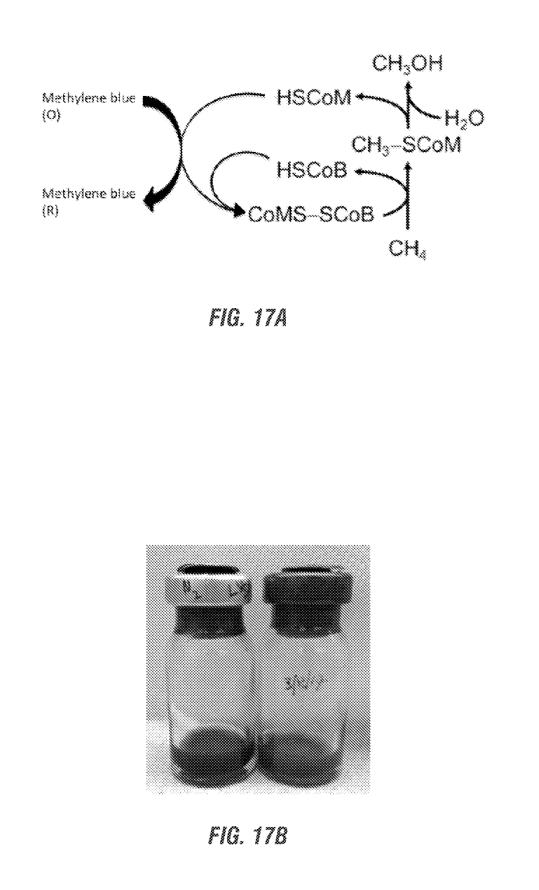

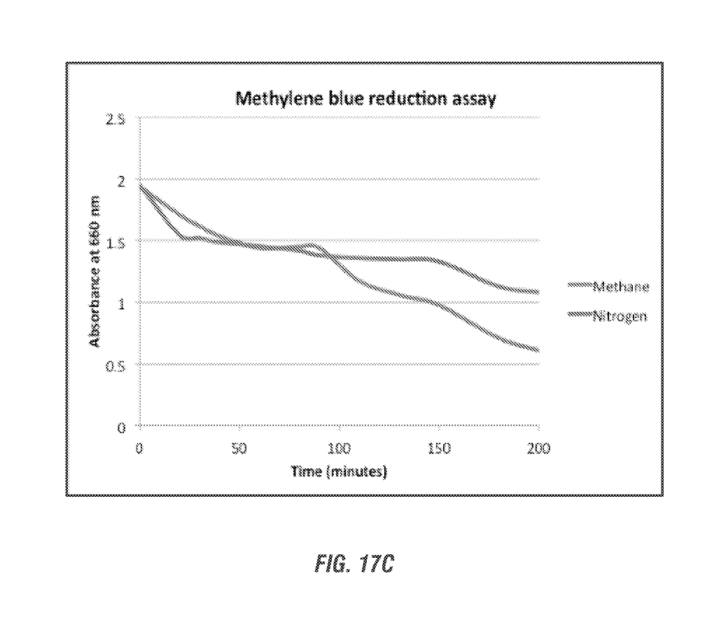

In order to determine the reversibility of methanogenic pathways, experiments were initiated using artificial electron acceptors for the oxidation of methane by Mcr, with the longer-term goal of co-culturing to enhance the rate of methane oxidation and produce reductant for production of liquid fuels and value-added products. When whole cells of M. acetivorans grown with methanol were incubated with methane and methylene blue, the electron acceptor was reduced as evidenced by loss of color (FIG. 17). The results indicate reversal of native Mcr supplying electrons to Hdr that reduced methylene blue also demonstrating reversal of Hdr. The results suggest that electrons can be delivered outside the cell to co-culture partners. The results also provide a convenient assay for reversed Mcr activity, facilitating modifications of Mcr leading to enhanced reverse activity.

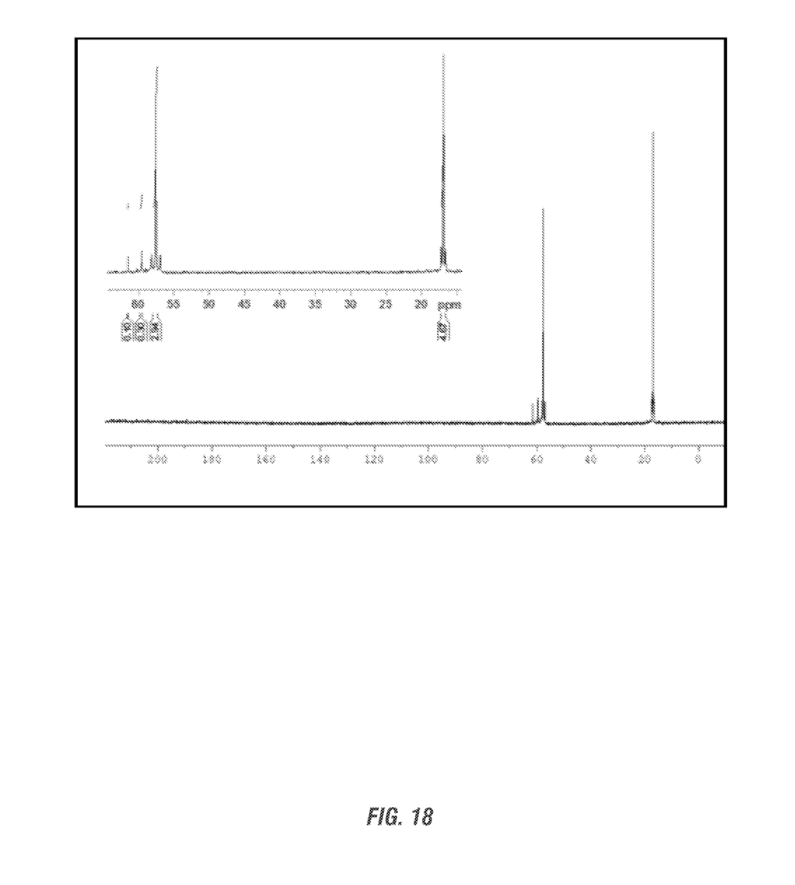

Next, methylene blue was replaced with 2-hydroxyphenazine, an analog of the physiological electron acceptor methanophenazine, and ¹³C-methane. NMR analyses of the assay mixture identified two methyl-containing compounds other than methane as products of the incubation, tentatively identified as acetate and methanol (FIG. 18).

The results further document the feasibility of co-culturing with an electron accepting partner. The results also document reversibility of the pathway, demonstrating the efficacy of processes for converting methane to liquid biofuels and other value-added products by monocultures of M. acetivorans.

Engineering Reversal of the Acetate-to-Methane Pathway to Obtain Baseline Rates of Acetyl-CoA Production

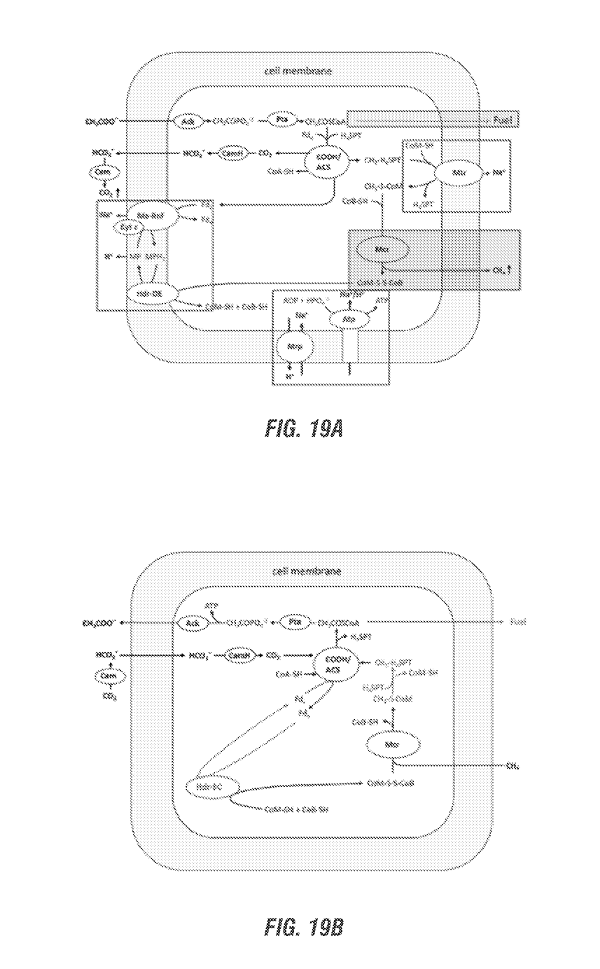

Conversion of methane to liquid fuels and value-added products depends on three modifications of the pathway shown in FIG. 19A leading to the pathway shown in FIG. 19B for production of acetyl-CoA. The modifications are: (1) reversal of the reaction catalyzed by methyl-coenzyme M methylreductase (Mcr) (pink box), (2) replacing membrane-bound complexes dependent on ion gradients (yellow boxes), and (3) diversion of acetyl-CoA to fuels and value-added products (green box).

The system was reconstituted in vitro in order to identify the electron carriers and the Hdr leading to optimization in a genetically engineered strain that by-passes the membrane-bound system reversing electron transport independent of an ion gradient (FIG. 3B).

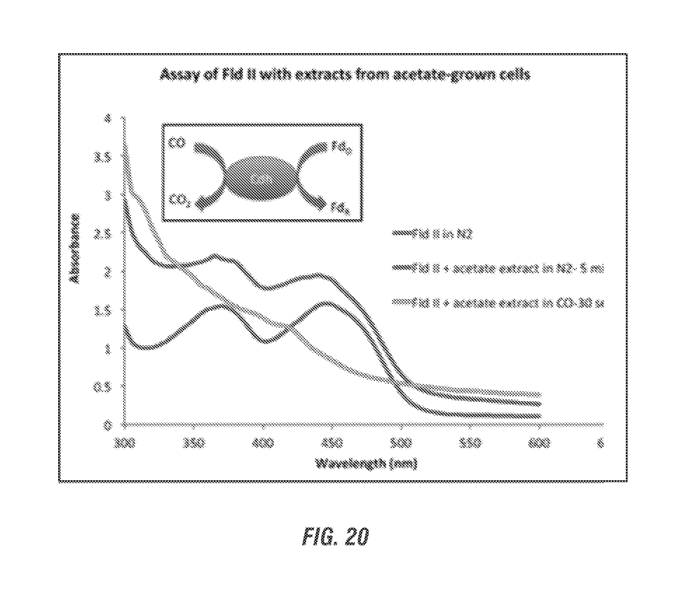

Reduction of a flavodoxin (FldI) coupled to oxidation of reduced Fd generated by the CO dehydrogenase/acetyl-CoA synthase (Cdh) was previously demonstrated in cell-free extract. Reduction of FdII is also coupled to oxidation of reduced Fd is further shown overexpression and purification of a second flavodoxin (FldII) (FIG. 20).

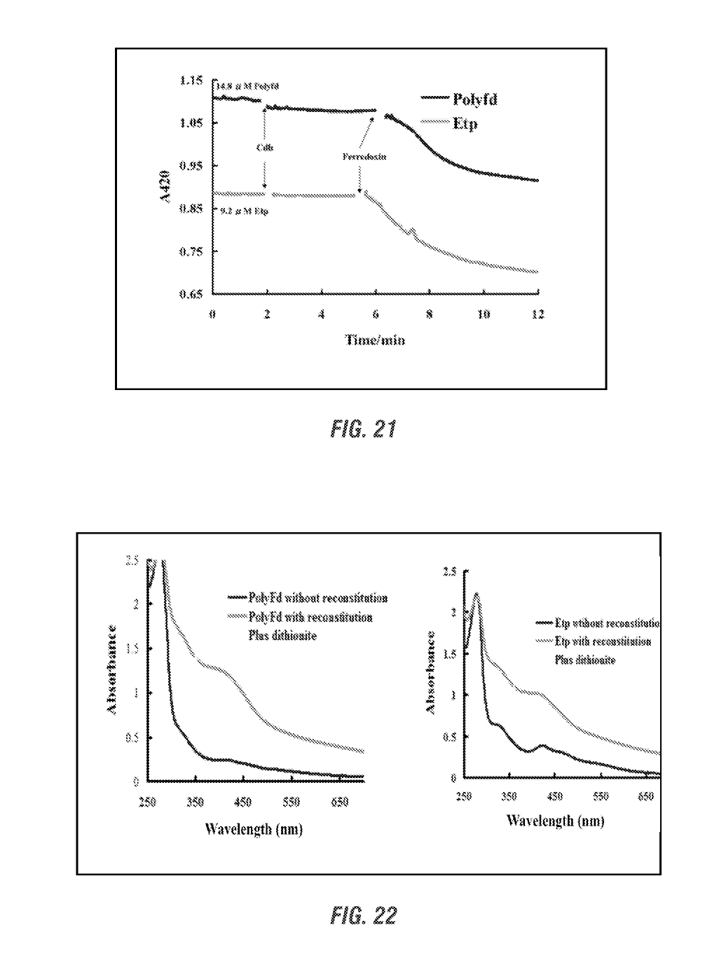

Previous work has also presented the purification of two soluble electron carriers (polyferredoxin, PolyFd; and electron transport protein, Etp). Reconstitution of these proteins with a full-compliment of iron-sulfur clusters is demonstrated in FIG. 21, and reduction of these proteins coupled to oxidation of Fd reduced with Cdh is shown in FIG. 22. The results demonstrate a role for one or both flavodoxins, Etp and PolyFd in the soluble electron transport system leading from Fd to the soluble Hdr. In summary, we have identified several electron carriers that are candidates for in vitro reconstitution of the soluble electron transport system.

Characterization of Promoter Structure for Optimization and Regulation of Engineered Pathways

MreA is a significant global regulator of methaneproducing pathways, and represents a target for optimization and regulation of engineered pathways reversing methanogenesis. The inventors have identified the MreA binding site in promoters to better design promoters most efficient in optimizing both expression and regulation of essential genes. Previously work using chromatin immunoprecipitation (ChIP) yielded results identifying a tentative binding site.

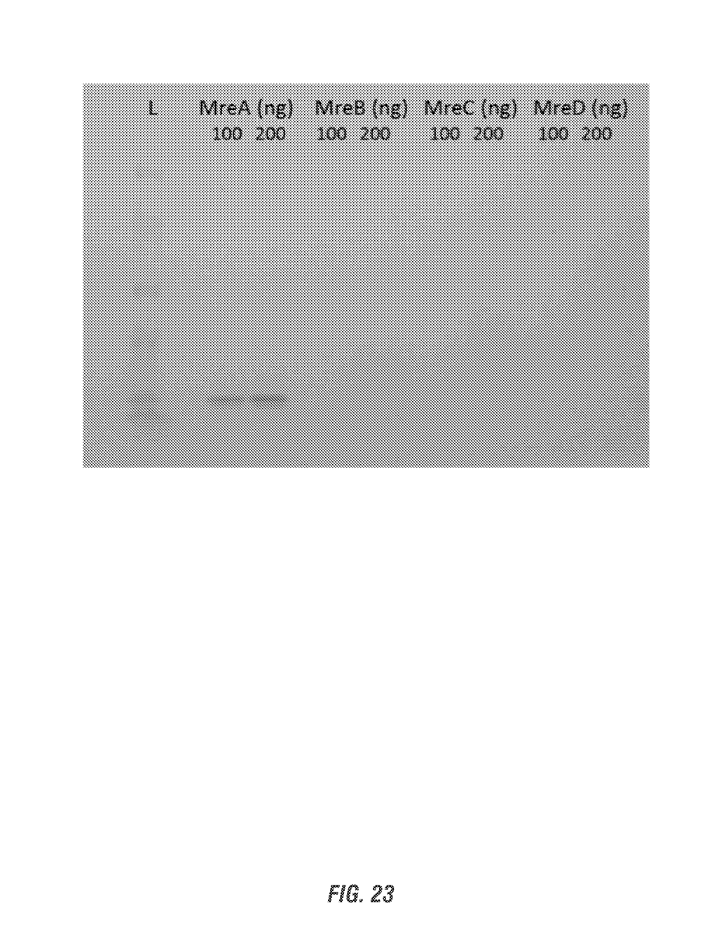

However, the genome encodes three MreA homologs with the potential to interact with MreA antisera confounding specificity of binding. Accordingly, all three homologs were overexpressed and purified, and showed that none cross react with MreA antisera (FIG. 23). The results confirm the binding site is specific for MreA, demonstrating a key aspect of expression and regulation of genes essential for the acetate-to-methane pathway and its reversal.

Growth of Metabolically Engineered Methanogens

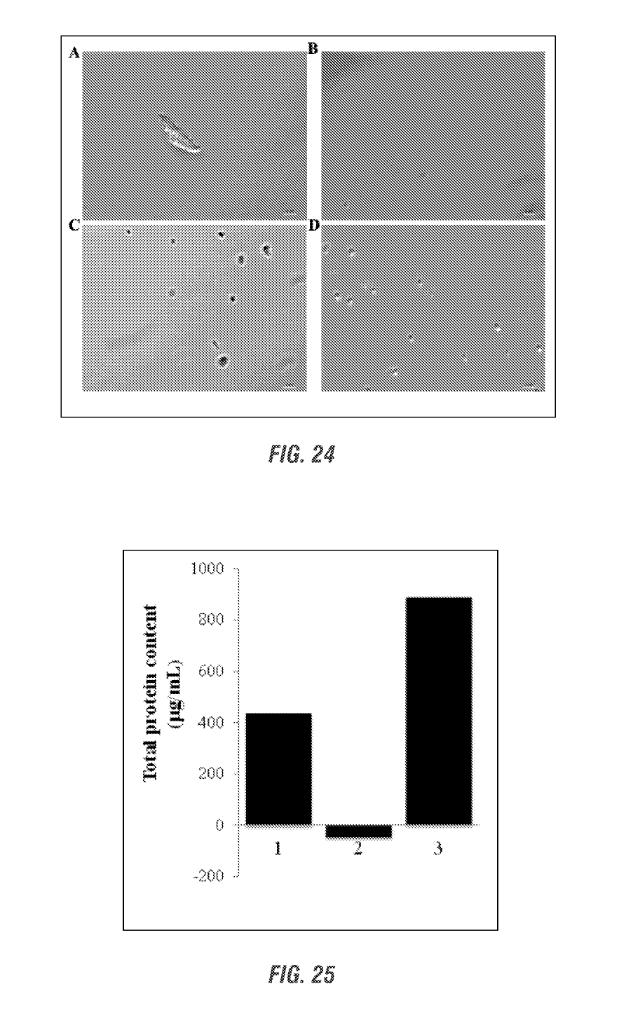

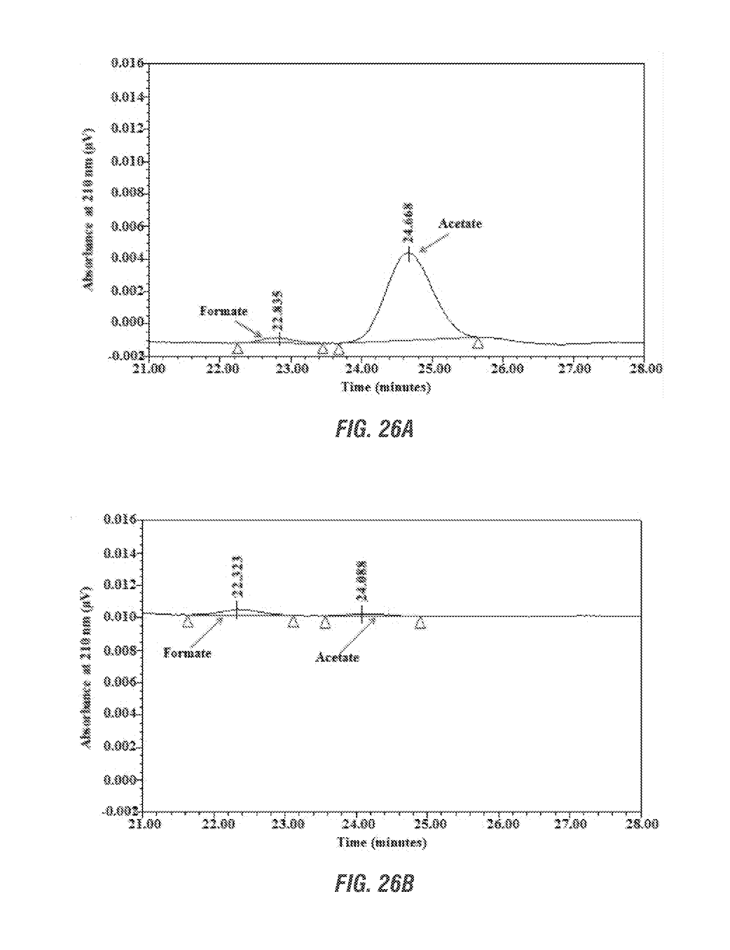

An acetate-grown and tetracycline-induced engineered M. acetivorans strain expressing Mcr was produced (C2A/pES1-MATmcr). The C2A strain was assessed initially for the ability to replicate. Using phase-contrast microscopy and hemocytometer counts, the cell number of the metabolically engineered strain, M. acetivorans C2A/pES1-MATmcr, increased by ˜10 fold after 30 days of incubation (FIG. 24). To further rule out the possibility of cross-contamination, we sequenced the 16s gene PCR-amplified from the methane-grown culture. Sequencing result confirmed the identity of cells growing on methane and 0.1 mM Fe³⁺ as M. acetivorans strain C2A. No PCR product was obtained when primers specific for bacterial 16s were used, and thus, confirming the homogeneity of the methane-grown culture. In addition, the ANME-1 mcr plasmid (pES1-MATmcr) could be PCR-amplified from the same methane-grown culture. This further validates the presence of pES1-MATmcr in M. acetivorans grown in methane and 0.1 mM Fe3+.

Increase in Total Protein

Using the bicinchoninic assay, a 450-fold increase in total protein for M. acetivorans C2A/pES1-ATmcr cultures grown in methane and 0.1 mM Fe³⁺ was obtained, in comparison to the negative control where growth was not observed (FIG. 25). This supports the presence of growth under this condition.

Methane Consumption

Gas chromatography was used to measure the level of methane in the headspace of tubes showing growth and no growth. After 46 days, 51% of the methane was consumed by cells grown on methane and 0.1 Mm FeCl₃. Hundreds of crimp-sealed 28-mL tubes showed no methane consumption, and methane was only consumed in tubes containing the engineered strain (e.g., no methane was consumed by strains with empty plasmids or no plasmid). Therefore, the wild-type strain is unable to consume methane, and methane consumption is a robust assay.

Methane, Not Bicarbonate or Cysteine in the Growth Medium, Provides the Carbon Source for Growth

The composition of the growth medium was further examined to negate the possibility of cells growing on a carbon source other than methane (190 mM). Computational approaches have also been used to check feasibilities of cells using certain media components as carbon sources, or as terminal electron acceptors. Consistent with data from these computational approaches, the present experimental results support that other major carbon sources in the growth medium, bicarbonate (45 mM) and cysteine (3.2 mM), are not sufficient to support the growth of ANME-1 Mcr-producing M. acetivorans. Trace vitamins found in the medium are below 0.005 mM, so we do not consider these as major carbon sources. Furthermore, we have yet to observe any growth of M. acetivorans (with and without ANME-1 mcr plasmid) when the headspace of the tube was filled with N₂ instead of CH₄. Omission of bicarbonate was also found to reduce the pH of the growth medium, while omission of cysteine renders the growth medium less reductive. In both cases, no growth was seen, despite adding methanol (the preferred carbon source of M. acetivorans) to the growth medium. Therefore, bicarbonate and cysteine are required for growth of M. acetivorans, but they act mainly as a buffering agent and a reducing agent, respectively.

Product Identification

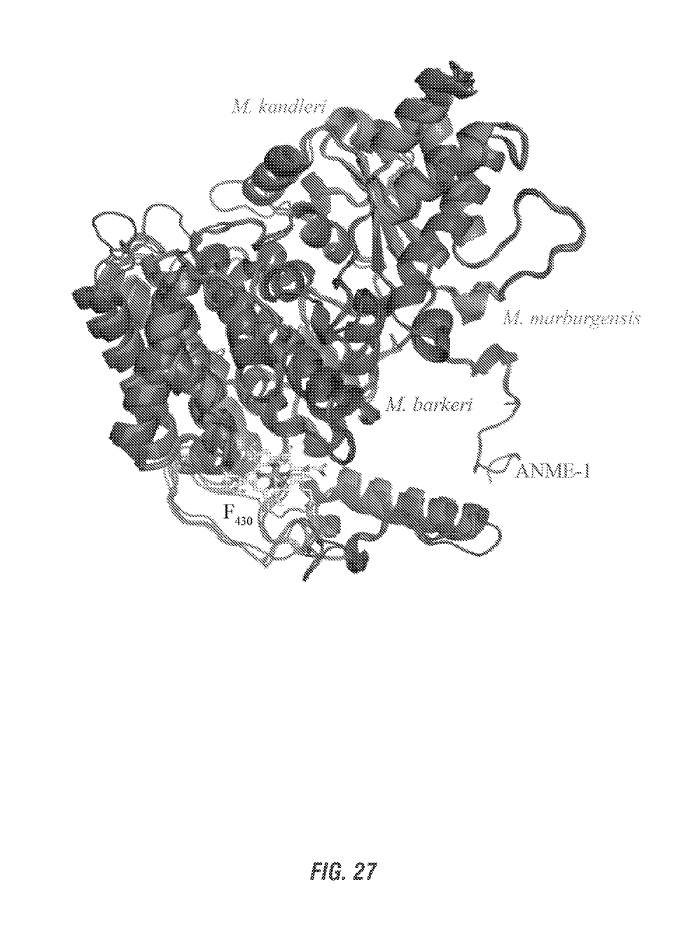

We identified several bio-products from growth on methane by M. acetivorans C2A/pES1-MATmcr. Gas chromatography and high performance liquid chromatography (HPLC) analyses were performed on methane-grown cultures from both liquid (culture medium) and gas (head-space) phases. Acetate (up to 6 mM), pyruvate (up to 10 μM), and hydrogen (up to 28 μM) were detected (see FIG. 27 for acetate detection) by the engineered strain; hence, the desirable intermediate acetate is produced by the engineered strain in large quantities. Formate was also detected at smaller amounts, but similar amounts of formate were found in samples without obvious signs of growth, so these low amounts of formate were likely formed by abiotic processes. Acetate, pyruvate, and hydrogen were not detected in as substantial amounts in tubes without obvious growth compared to tubes found with growth, so these were not made by abiotic processes.

To corroborate our results, pyruvate was also detected by liquid-chromatography-electrospray ionization-tandem mass spectrometry (LC-ESI-MS) analysis of the culture supernatant of the engineered strain (M. acetivorans/pES1-MATmcr) and the wild-type M. acetivorans. In comparison to the wild-type M. acetivorans strain grown on methanol (the preferred substrate), the methane-grown, engineered strain secreted 40 fold higher pyruvate. This indicates pyruvate secretion is specific to the engineered strain growing on methane. Acetate was not detected by LC-ESI-MS, as the molecular weight of acetate is smaller than the detection limit of LC-ESI-MS (˜80 g/mol).

Methane Consumption by Samples Inoculated with Sludge

Growth selection experiments were begun by incubating activated sludge from anaerobic digesters of waste-water treatment plants as an alternative approach for converting methane into biofuels. After 16 days of incubation, growth was seen at various concentrations of SO₄²⁻ (ranging from 0.01 to 100 mM) and with 10 mM Fe³⁺. Gas chromatography analysis of the methane remaining in the headspace of these cultures indicated promising amounts of methane being consumed from 9% to 69%. PCR amplification of the 16s genes from the sludge samples indicate the presence of both archaea and bacteria. The dominating species appears to be an unculturable Methanomicrobiales archaeon based on preliminary sequencing analysis of the 16s genes. However, upon enriching the consortium present in the activated sludge (i.e., by subculturing into new growth medium), methane was only moderately consumed (up to 10%) after 41 days.

Computational Protein Engineering

Overview

Within the framework of producing liquid fuels from methane, computational protein engineering is used to improve the activity of rate-limiting enzymes along the methaneto-acetate pathway, namely methyl-coenzyme M reductase (Mcr). Factor F430 is a nickel-centered tetrapyyrole cofactor in Mcr. All known methanogenic archaea Mcrs contain an unmodified F430. In contract, an Mcr homolog from a consortia of methanotrophic archaea (ANME Mcr) consists of a methylthiolated F430. This key discrepancy between cofactors may be responsible for the methanotrophic activity in ANME Mcr. Since the methylthio moiety lies at the exterior of the F430 macrocycle, this modification may be necessary to lock the cofactor into the proper geometry within the active site. Alteration of the cofactor specificity of ANME Mcr to the unmodified F430 was investigated, and expression of this mutant protein in M. acetivorans, where the unmodified cofactor is accessible.

Algorithmic Inputs

Iterative Protein Redesign & Optimization (IPRO) [1] procedures were used to help identify mutations that improve ANME Mcr activity. IPRO relies on molecular statics calculations, and it was thus necessary to fully parameterize the system before any calculations were performed. The molecules that were parameterized include coenzyme M, coenzyme B, and the unmodified Ni(I) F430. Parameterization was largely performed using CGENFF version 2b8 [2, 3], which obtains parameters from a database of analogous molecules. The validity of analogies made using CGENFF are quantified using penalty scores. Mulliken population analysis from Ni(I) unmodified F430 quantum mechanics calculations were used to estimate partial atomic charges for atoms with high CGENFF penalty scores. The optimized geometry of the Ni(I) unmodified cofactor was also used to adjust highly penalized equilibrium bond lengths and bond angles. All adjusted partial charges were a weighted combination of the CGENFF value and the QM value, where the value of the weight was dependent upon the penalty score (higher penalty, higher contribution from quantum mechanics).

In addition to small molecule parameterization, IPRO also requires design position selection. A design position is an amino acid that is permitted to mutate away from wild-type such that binding to a target molecule is improved. Mcrs from highly diverse methanogenic archaea display high sequence conservation (61-69% identity), and these Mcrs and are also largely homologous with ANME Mcrs (˜50% identity). Since the ANME Mcr sequence is largely conserved, destruction of catalytic activity by mutating an essential residue was to be avoided. Structure and sequence alignments were used to find residues that were highly conserved amongst methanogenic Mcrs but more diverse in ANME Mcrs.

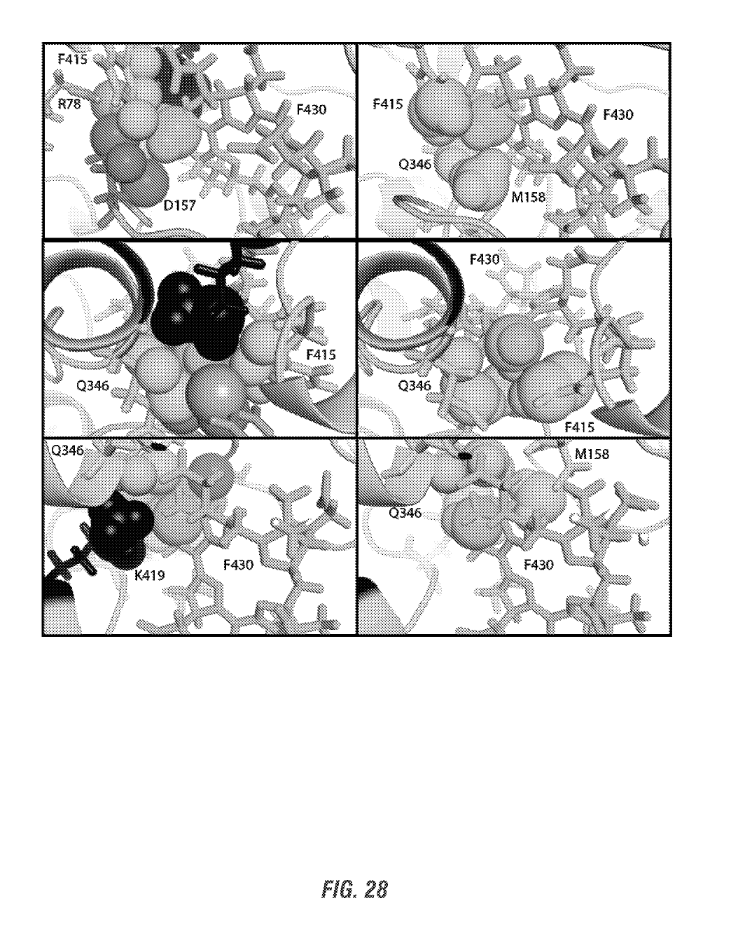

Structure alignments were performed by aligning the homologous regions of ANME-1, Methanothermobacter marburgensis, Methanopyrus kandleri, and Methanosarcina barkeri Mcrs' α subunits. Homologous regions were identified using BLAST and were then aligned by minimizing the root mean squared deviation (RMSD) between the backbones of the homologous regions. For consistency, all Mcrs were aligned to the coordinates corresponding to ANME-1 Mcr. Following the structure alignment protocol, the backbone atom RMSD between the a subunits of ANME-1 Mcr and the Mcrs of M. marburgensis, M. kandleri, and M. barkeri were 1.12, 1.20, and 3.85 Å, respectively. The alignment of these α subunits can be seen in FIG. 28.

Sequence alignment was performed using Clustal-Omega version 1.2.1 on both Mcr and ANME Mcr sequences. P07962, P22948, A4PJ22, D3E050, P12971, P11558, O27232, Q49605, Q58256, P11559, P07961, Q6LWZ5 are the Uniprot sequence IDs for methanogenic Mcrs. The sequence IDs for the methanotrophic ANME Mcrs are Q6VUA6, Q64E03, Q64EA1, Q648C5, Q64D16, D1JBK4, Q6MZD1, Q64CB7, Q64AN3, Q64EF1, Q649Z5, and Q64DN6. Sequence ID D1JBK4 is the primary structure for the lone crystallized ANME Mcr (ANME-1 Mcr [5], see FIG. 27). Two sequence alignments were performed. The first alignment was amongst all ANME Mcrs, and the second was amongst the methanogenic Mcrs in addition to sequence D1JBK4. Positions within ANME-1 Mcr that were highly conserved (>75%) amongst methanogenic Mcrs but varied amongst (<45%) ANME Mcrs were identified. 77 positions met this criteria and may suggest possible locations in the structure that can reverse the native Mcr reaction.

Sequence alignment, distance from the active site, and literature all contributed to the eventual design position choice. The sequence alignment comparison was used and the nine closest residues to the modified portion of the cofactor were chosen. In addition to these nine residues, a tenth design position (V419) was selected as it was suggested to accommodate the modified cofactor by others. The ten selected design positions were Q72, L77, M78, N90, P149, I154, H157, H414, V419, and C423.

IPRO was used to redesign ANME Mcr to tightly bind the unmodified cofactor. If the purpose of the methylthiolation is to lock the cofactor into its proper orientation, then we can introduce mutations to ANME Mcr to change cofactor specificity. The IPRO trajectory was run for 2000 iterations using the standard CHARMM energy terms without the use of solvation. We can neglect solvation here because the binding of CoB blocks the ability of water to reach the active site. Neglecting solvation decreases the computational time required for IPRO and also decreases the energetic variance between sequentially identical mutants. ANME Mcr was modeled by only using the two α subunits immediately adjacent to the unmodified cofactor. The β and γ subunits were removed since they are sufficiently distant from the active site that they would not affect nonbonded interactions within the active site. The IPRO runs additionally contained weak position restraints that limited how much atoms could move from the initial ANME-1 Mcr structure. The model furthermore contained two restraints that limited the distances of the atoms coordinated to the nickel of Factor F430. IPRO was adjusted to avoid tendencies to mutate to glycine (through incorporation of a softening term) and charged residues (by matching the electrostatic contribution to the total energy in Rosetta). The designs predicted to improve binding affinity for the unmodified cofactor are presented in Table 5. From Table 5, I154N, V419K, and C423N are all completely conserved. V419K seems to be a critical mutation suggested by IPRO as it was also conserved from another set of results (data not shown). The structure of V419K also suggests efficient packing around C172 (see FIG. 28), which is the carbon atom where the methylthiolation takes place in the modified cofactor. V419K does not create any hydrogen bonds and does not create any strong electrostatic interactions (a weak interaction may be formed with one of the short chain fatty acids of F430). I154N also contacts the cofactor but is not proximal to C172. C423N does not directly contact the cofactor but helps stabilize the conformation of the loop containing V419K. However, C423 may be involved in an electron shuttling mechanism for the enzyme. Analysis of the Mutant 1 (i.e., top mutant from Table 5) structure showed that M78R (90% conservation among all results) and H157D (60% conservation) also contact C172 (see FIG. 28). Thus, these mutations are likely to alter ANME-1 cofactor specificity.

TABLE 5
Top ten mutants for improving binding to Cofactor F430. Changes from
wild-type are shown in red. “Mutant” is abbreviated as “Mut.”. Energies are calculated in
CHARMM.

| Mut. | Q72 | L77 | M78 | N90 | P149 | I154 | H157 | H414 | V419 | C423 | Energy |
|---|---|---|---|---|---|---|---|---|---|---|---|
| 1 | Q | A | R | A | A | N | D | G | K | N | −114.86 |
| 2 | Q | A | R | R | A | N | D | G | K | N | −114.17 |
| 3 | Q | D | R | R | A | N | N | G | K | N | −111.78 |
| 4 | Q | A | R | R | A | N | D | A | K | N | −111.39 |
| 5 | Q | D | R | R | A | N | D | G | K | N | −110.49 |
| 6 | Q | D | R | R | A | N | D | A | K | N | −109.49 |
| 7 | Q | A | K | R | A | N | D | A | K | N | −109.28 |
| 8 | Q | D | R | R | A | N | N | C | K | N | −108.05 |
| 9 | M | D | R | R | E | N | N | C | K | N | −107.77 |
| 10 | M | D | R | R | Q | N | N | C | K | N | −107.71 |

Genome-Scale Metabolic Modeling

Model Development

An extended genome-scale metabolic model for M. acetivorans, iMAC865, was constructed by adding reactions from a previously published model (iVS941) to a more recently published model, iMB745. The integrated model contains 865 genes, 827 reactions, and 712 metabolites. 117 GPRs were only found in iVS941 model, and 194 GPRs were only found in iMB745 model. 277 GPRs were found in both models and were in agreement, while 38 reactions found in both models had differing gene associations. All 38 GPRs were revised and corrected using a list of 781 newly revised gene re-annotations (Ferry and co-workers, unpublished data) along with the KEGG database and relevant literature. 10 of these reactions were involved in the methanogenesis pathway. 13 reactions required elemental and charge rebalancing due to missing or wrong chemical formulae. Three of these reactions were in the methanogenesis pathway. iMAC865 more accurately predicts biomass yields on methanol and acetate as compared to iMB745 as shown in Table 6.

TABLE 6
Biomass yield predictions by iMAC865. Comparison
of biomass yield predictions for iMB745 and
iMAC865 against the experimentally reported yields.

| | Observed Growth | Predicted Growth Yield (g | |
|---|---|---|---|
| | Yield (g cDW/mmol | cDW/mmol substrate) | |
| Substrate | substrate) | iMB745 | iMAC865 |
| Methanol | 5.2 | 4.0 | 5.26 |
| Acetate | 2.4 | 3.0 | 2.6 |

Incorporation of Substrate Specific Protein Measurements

Along with updates to the genome-scale model, the Maranas lab has incorporated regulatory interactions via a regulatory gene-protein-reaction (R-GPR) approach. This approach modifies certain GPRs and removes select reactions in order to align with the protein expression profiles for different substrates. M. acetivorans has been shown to have differential transcriptome and proteome profiles depending on the substrate it utilizes. This approach was used for the methanol/acetate protein expression dataset published by Li et al. The dataset contained quantitative protein levels for over 250 genes of M. acetivorans grown on acetate and methanol.

The ratio of protein abundance for acetate to methanol or methanol to acetate was calculated and any genes that had values below a cutoff of 0.25 were assigned to a set G. The reactions with GPRs that had genes within set G were then reevaluated assuming that all genes in set G were knocked out. Those reactions that would still be active had their GPRs modified to account for the loss of these genes. Those reactions that could not exist without the genes in set G were added to set R. The maximum number of reactions in set R that could be removed without dropping the biomass yield below the experimentally reported value was identified.

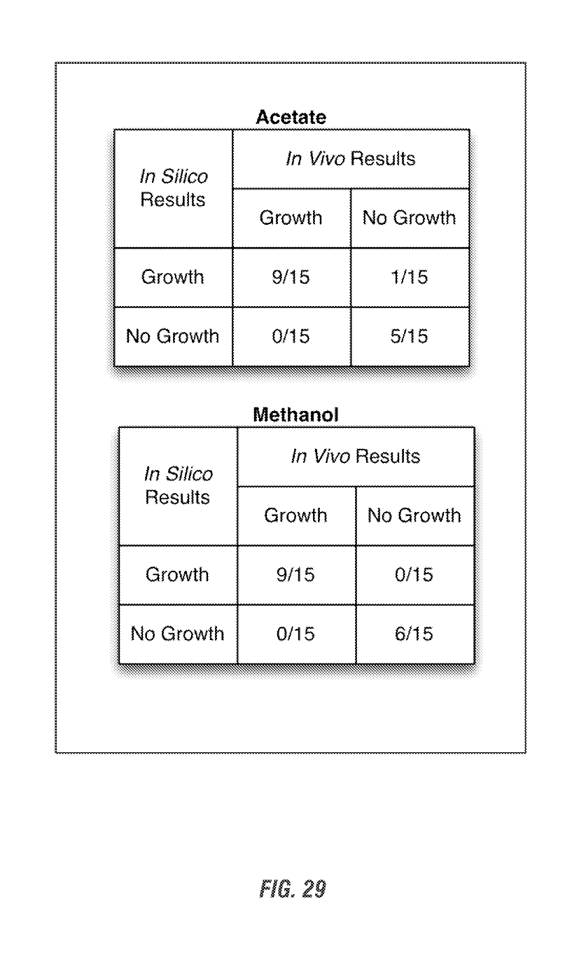

The R-GPR approach suggested the removal of 34 out of a possible 47 reactions when the sole carbon source was acetate to keep the in silico yield at the in vivo yield. 45 additional reactions had their GPRs modified. When the carbon source was methanol, 5 out of 8 possible reactions were removed and 12 GPRs were modified. These changes led to an increase from 90% to 96.7% for the correct prediction of gene deletion mutants grown on acetate and methanol (FIG. 29).

These R-GPR modifications may be implemented again on the M. acetivorans model when the final products from the reverse methanogenesis pathway are determined, as such products could trigger similar regulatory responses as when they are supplied as substrates.

The Reverse Methanogenesis Pathway in the Genome-Scale Model

The iMAC865 model was modified in order to test the hypothesis that pure cultures of M. acetivorans can oxidize methane through the reversal of the methanogenesis pathway (FIG. 13). A new reaction was added to the iMAC865 model, which is identical to the methyl coenzyme M reductase but acts in the reverse direction. The original MCR reaction was switched-off for growth on methane. This new reaction describes the anaerobic methanotrophic (ANME) methyl coenzyme M reductase that was included in the M. acetivorans C2A/pES1-MATmcr. Since the energy and sodium/proton ion translocation requirements for M. acetivorans growing on methane are not known, we propose that cells might produce ATP mainly through substrate-level phosphorylation and cofactors might be regenerated by soluble enzymes. This combined with no proton gradient generated to drive the membrane bound reactions in the opposite direction, reactions corresponding to the multi-unit membrane-bound enzyme complexes Mtr, Hdr, Rnf and Fpo were switched off in the iMAC865 model during growth on methane (enzymes shown in gray in FIG. 16). These reactions pump sodium and proton ions in and out of the cells as part of the electron transport system in M. acetivorans. To compensate for the loss of membrane-bound Mtr, a soluble methyltransferase was added to the reconstruction, which does not require sodium ion translocation for M. acetivorans cells grown on CO. Cytoplasmic heterodisulfide (HdrABC) was shown to be involved in methylotrophic methanogenesis and aceticlastic methanogenesis. Therefore, the reaction representing this soluble heterodisulfide reductase was kept in the model while the membrane-bound HDR reaction was switched off.

Therefore various methods, compositions, bioengineered organisms, and systems have been disclosed relating to making biofuels, including developing bioengineered organisms, the resulting bioengineered organisms, method for identifying pathways, and other methods, compositions, and systems. Although specific examples have been provided throughout, numerous variations, options, and alternatives are contemplated including variations in the particular pathways used, the manner in which the pathways are identified, the type of intermediates produced, and the type of fuel produced. The present invention is not to be limited to the specific disclosure provided herein as numerous options, variations, and alternatives are contemplated.

### REFERENCES

All references provided herein are hereby incorporated by reference in their entirety.
1. AbuOun, M., P. F. Suthers, G. I. Jones, B. R. Carter, M. P. Saunders, C. D. Maranas, M. J. Woodward, and M. F. Anjum. 2009. Genome scale reconstruction of a Salmonella metabolic model: comparison of similarity and differences with a commensal Escherichia coli strain. J Biol Chem 284:29480-29488.
2. Alber, B. E., and J. G. Ferry. 1994. A carbonic anhydrase from the archaeon Methanosarcina thermophila. Proc. Natl. Acad. Sci. USA 91:6909-6913.
3. Apolinario, E. E., K. M. Jackson, and K. R. Sowers. 2005. Development of a plasmid-mediated reporter system for in vivo monitoring of gene expression in the archaeon Methanosarcina acetivorans. Applied and Environmental Microbiology 71:4914-4918.
4. Bansal, T., D. Englert, J. Lee, M. Hegde, T. K. Wood, and A. Jayaraman. 2007. Differential effects of epinephrine, norepinephrine, and indole on Escherichia coli O157:H7 chemotaxis, colonization, and gene expression. Infect. Immun. 75:4597-4607.
5. Barriault, D., M.-M. Plante, and M. Sylvestre. 2002. Family Shuffling of a Targeted bphA Region To Engineer Biphenyl Dioxygenase. J. Bacteriol. 184:3794-3800.
6. Bianchi, V., P. Reichard, R. Eliasson, E. Pontis, M. Krook, H. Jornvall, and E. Haggard-Ljungouist. 1993. Escherichia coli ferredoxin NADP⁺ reductase: activation of E. coli anaerobic ribonucleotide reduction, cloning of the gene (fpr), and overexpression of the protein. J. Bacteriol. 175:1590-1595.
7. Boiangiu, C. D., E. Jayamani, D. Brugel, G. Herrmann, J. Kim, L. Forzi, R. Hedderich, I. Vgenopoulou, A. J. Pierik, J. Steuber, and W. Buckel. 2005. Sodium ion pumps and hydrogen production in glutamate fermenting anaerobic bacteria. J Mol Microbiol Biotechnol 10:105-119.
8. Burgard, A. P., P. Pharkya, and C. D. Maranas. 2003. Optknock: a bilevel programming framework for identifying gene knockout strategies for microbial strain optimization. Biotechnol Bioeng 84:647-657.
9. Bylina, E. J., W. J. Coleman, M. R. Dilworth, S. J. Robles, M. A. Tanner, M. M. Yang, and D. C. Youvan. 2000. Solid-phase enzyme screening. ASM News 66. :211-217.
10. Canada, K. A., S. Iwashita, H. Shim, and T. K. Wood. 2002. Directed Evolution of Toluene ortho-Monooxygenase for Enhanced 1-Naphthol Synthesis and Chlorinated Ethene Degradation. Journal of Bacteriology 184:344-349.
11. Canada, K. A., S. Iwashita, H. Shim, and T. K. Wood. 2002. Directed evolution of toluene ortho-monooxygenase for enhanced 1-naphthol synthesis and chlorinated ethene degradation. J. Bacteriol. 184:344-349.
12. Ceccarelli, E. A., A. M. Viale, A. R. Krapp, and N. Carrillo. 1991. Expression, assembly, and processing of an active plant ferredoxin-NADP+ oxidoreductase and its precursor protein in Escherichia coli. J. Biol. Chem. 266:14283-142877.
13. Cheng, S., D. Xing, D. F. Call, and B. E. Logan. 2009. Direct Biological Conversion of Electrical Current into Methane by Electromethanogenesis. Environmental Science & Technology 43:3953-3958.
14. Crameri, A., and W. P. Stemmer. 1995. Combinatorial multiple cassette mutagenesis creates all the permutations of mutant and wild-type sequences. BioTech. 18:194-196.
15. Crameri, A., E. A. Whitehorn, E. Tate, and W. P. Stemmer. 1996. Improved green fluorescent protein by molecular evolution using DNA shuffling. Nat Biotechnol 14:315-319.
16. Domka, J., J. Lee, T. Bansal, and T. K. Wood. 2007. Temporal gene-expression in Escherichia coli K-12 biofilms. Environ. Microbiol. 9:332-346.
17. Domka, J., J. Lee, and T. K. Wood. 2006. YliH (BssR) and YceP (BssS) regulate Escherichia coli K-12 biofilm formation by influencing cell signaling. Appl. Environ. Microbiol. 72:2449-2459.
18. Dunbrack, R. L., Jr., and M. Karplus. 1993. Backbone-dependent rotamer library for proteins. Application to side-chain prediction. Journal of molecular biology 230:543-574.
19. Dunbrack, R. L., Jr., and M. Karplus. 1994. Conformational analysis of the backbone-dependent rotamer preferences of protein sidechains. Nature structural biology 1:334-340.
20. Eggen, R. I. L., A. C. M. Geerling, A. B. P. Boshoven, and W. M. Devos. 1991. Cloning, sequence analysis, and functional expression of the acetyl coenzyme A synthetase gene from Methanothrix soehngenii in Escherichia coli. Journal of Bacteriology 173:6383-6389.
21. Fazelinia, H., P. C. Cirino, and C. D. Maranas. 2009. OptGraft: A computational procedure for transferring a binding site onto an existing protein scaffold. Protein Sci 18:180-195.
22. Ferry, J. G. 2011. Acetate kinase and phosphotransacetylase. Methods Enzymol. 494:219-231.
23. Ferry, J. G. 1995. CO dehydrogenase. Annu. Rev. Microbiol. 49:305-333.
24. Ferry, J. G. 2010. How to make a living exhaling methane. Annu. Rev. Microbiol. 64:453-473.
25. Ferry, J. G., and K. A. Kastead. 2007. Methanogenesis, p. 288-314. In R. Cavicchioli (ed.), Archaea: Molecular Cell Biology. ASM Press, Washington, D.C.
26. Finkel, M. L., and A. Law. 2011. The Rush to Drill for Natural Gas: A Public Health Cautionary Tale. American Journal of Public Health 101:784-785.
27. Fishman, A., Y. Tao, W. Bentley, and T. K. Wood. 2004. Protein Engineering of Toluene 4-Monooxygenase of Pseudomonas mendocina KR1 for Synthesizing 4-Nitrocatechol from Nitrobenzene. Biotechnology and Bioengineering 87:779-790.
28. Fishman, A., Y. Tao, L. Rui, and T. K. Wood. 2005. Controlling the regiospecific oxidation of aromatics via active site engineering of toluene para-monooxygenase of Ralstonia pickettii PKO1. J Biol Chem 280:506-514.
29. Fishman, A., Y. Tao, G. Vardar, L. Rui, and T. K. Wood. 2006. Controlling regiospecific oxidation of aromatics and the degradation of chlorinated aliphatics via active site engineering of toluene monooxygenases, p. 506-514. In J.-L. Ramos and R. C. Vevesque (ed.), Pseudomonas 4: Molecular Biology of Emerging Issues, vol. 280. Springer, Netherlands.
30. Fishman, A., Y. Tao, and T. K. Wood. 2004. Physiological relevance of successive hydroxylations of toluene by toluene para-monooxygenase of Ralstonia pickettii PKO1. Biocatalysis and Biotransformation 22:283-289.
31. Gottschalk, G., and R. K. Thauer. 2001. The Na⁺ translocating methyltransferase complex from methanogenic archaea. Biochimica et biophysica acta 1505:28-36.
32. Grahame, D. A. 2003. Acetate C—C bond formation and decomposition in the anaerobic world: the structure of a central enzyme and its key active-site metal cluster. Trends Biochem. Sci. 28:221-224.
33. Hedderich, R., N. Hamann, and M. Bennati. 2005. Heterodisulfide reductase from methanogenic archaea: a new catalytic role for an iron-sulfur cluster. Biol. Chem. 386:961-970.
34. Herzberg, M., I. K. Kaye, W. Peti, and T. K. Wood. 2006. YdgG (TqsA) Controls Biofilm Formation in Escherichia coli K-12 by Enhancing Autoinducer 2 Transport. J. Bacteriol. 188:587-598.
35. Hong, S. H., M. Hegde, J. Kim, X. Wang, A. Jayaraman, and T. K. Wood. 2012. Synthetic quorum-sensing circuit to control consortial biofilm formation and dispersal in a microfluidic device. Nature Communications 3:613.
36. Hong, S. H., J. Lee, and T. K. Wood. 2010. Engineering global regulator Hha of Escherichia coli to control biofilm dispersal. Microb. Biotechnol. 3:717-728.
37. Hong, S. H., X. Wang, and T. K. Wood. 2010. Controlling biofilm formation, prophage excision, and cell death by rewiring global regulator H—NS of Escherichia coli. Microb. Biotechnol. 3:344-356.
38. Hovey, R., S. Lentes, A. Ehrenreich, K. Salmon, K. Saba, G. Gottschalk, R. P. Gunsalus, and U. Deppenmeier. 2005. DNA microarray analysis of Methanosarcina mazei Go1 reveals adaptation to different methanogenic substrates. Mol Genet Genomics 273:225-239.
39. http://webbook.nist.gov/chemistry/.
40. Huo, F.-J., Y.-Q. Sun, J. Su, J.-B. Chao, H.-J. Zhi, and C.-X. Yin. 2009. Colorimetric Detection of Thiols Using a Chromene Molecule. Organic Letters 11:4918-4921.
41. Inokuma, K., Y. Nakashimada, T. Akahoshi, and N. Nishio. 2007. Characterization of enzymes involved in the ethanol production of Moorella sp. HUC22-1. Arch. Microbiol. 188:37-45.
42. Jahng, D., and T. K. Wood. 1994. Trichloroethylene and chloroform degradation by a recombinant Pseudomonad expressing soluble methane monooxygenase from Methylosinus trichosporium OB3b. Appl. Environ. Microbiol. 60:2473-2482.
43. Joo, H., Z. Lin, and F. H. Arnold. 1999. Laboratory Evolution of Peroxide-Mediated Cytochrome P450 Hydroxylation. Nature 399:670-673.
44. Keenan, B. G., T. Leungsakul, B. F. Smets, M.-A. Mori, D. E. Henderson, and T. K. Wood. 2005. Protein Engineering of the Archetypal Nitroarene Dioxygenase of Ralstonia sp. strain U2 for Activity on Aminonitrotoluenes and Dinitrotoluenes Through Alpha-Subunit Residues Leucine 225, Phenylalanine 350, and Glycine 407. J. Bacteriol. 187.
45. Keenan, B. G., T. Leungsakul, B. F. Smets, and T. K. Wood. 2004. Saturation Mutagenesis of Burkholderia cepacia R34 2,4-Dinitrotoluene Dioxygenase at DntAc Valine 350 for Synthesizing Nitrohydroquinone, Methylhydroquinone, and Methoxyhydroquinone. Applied and Environmental Microbiology 70:3222-3231.
46. Khoury, G. A., H. Fazelinia, J. W. Chin, R. J. Pantazes, P. C. Cirino, and C. D. Maranas. 2009. Computational design of Candida boidinii xylose reductase for altered cofactor specificity. Protein Sci 18:2125-2138.
47. Kuhlman, B., G. Dantas, G. C. Ireton, G. Varani, B. L. Stoddard, and D. Baker. 2003. Design of a novel globular protein fold with atomic-level accuracy. Science 302:1364-1368.
48. Kumamaru, T., H. Suenaga, M. Mitsuoka, T. Watanabe, and K. Furukawa. 1998. Enhanced Degradation of Polychlorinated Biphenyls by Directed Evolution of Biphenyl Dioxygenase. Nature Biotechnology 16:663-666.
49. Kumar, A., P. F. Suthers, and C. D. Maranas. 2012. MetRxn: a knowledgebase of metabolites and reactions spanning metabolic models and databases. BMC Bioinformatics 13:6.
50. Kumar, V. S., and C. D. Maranas. 2009. GrowMatch: an automated method for reconciling in silico/in vivo growth predictions. PLoS Comput Biol 5:e1000308.
51. Lee, J., T. Bansal, A. Jayaraman, W. E. Bentley, and T. K. Wood. 2007. Enterohemorrhagic Escherichia coli biofilms are inhibited by 7-hydroxyindole and stimulated by isatin. Appl. Environ. Microbiol. 73:4100-4109.
52. Lee, J., A. Jayaraman, and T. K. Wood. 2007. Indole is an inter-species biofilm signal mediated by SdiA. BMC Microbiol. 7:42.
53. Lee, J., T. Maeda, S. H. Hong, and T. K. Wood. 2009. Reconfiguring the quorum-sensing regulator SdiA of Escherichia coli to control biofilm formation. Appl. Environ. Microbiol. 75:1703-1716.
54. Lee, J., R. Page, R. García-Contreras, J.-M. Palermino, X.-S. Zhang, O. Doshi, T. K. Wood, and W. Peti. 2007. Structure and Function of the Escherichia coli Protein YmgB: A Protein Critical for Biofilm Formation and Acid Resistance. J Mol Biol 373:11-26.
55. Lee, J., X.-S. Zhang, M. Hegde, W. E. Bentley, A. Jayaraman, and T. K. Wood. 2008. Indole cell signaling occurs primarily at low temperatures in Escherichia coli. ISME J 2:1007-1023.
56. Lessner, D. J., L. Lhu, C. S. Wahal, and J. G. Ferry. 2010. An engineered methanogenic pathway derived from the domains Bacteria and Archaea, p. e00243-00210, mBio, vol. 1.
57. Lessner, D. J., L. Li, Q. Li, T. Rejtar, V. P. Andreev, M. Reichlen, K. Hill, J. J. Moran, B. L. Karger, and J. G. Ferry. 2006. An unconventional pathway for reduction of CO₂ to methane in CO-grown Methanosarcina acetivorans revealed by proteomics. Proc. Natl. Acad. Sci. U.S.A. 103:17921-17926.
58. Lessner, D. J., L. Li, Q. Li, T. Rejtar, V. P. Andreev, M. Reichlen, K. Hill, J. J. Moran, B. L. Karger, and J. G. Ferry. 2006. An unconventional pathway for reduction of CO₂ to methane in CO-grown Methanosarcina acetivorans revealed by proteomics. Proc. Natl. Acad. Sci. U.S.A. 103:17921-17926.
59. Leungsakul, T., G. R. Johnson, and T. K. Wood. 2006. Protein Engineering of the 4-Methyl-5-Nitrocatechol Monooxygenase from Burkholderia sp. strain DNT for Enhanced Degradation of Nitroaromatics. Applied and Environmental Microbiology 72:3933-3939.
60. Leungsakul, T., B. G. Keenan, H. Yin, B. F. Smets, and T. K. Wood. 2005. Saturation Mutagenesis of 2,4-DNT Dioxygenase of Burkholderia sp. strain DNT for Enhanced Dinitrotoluene Degradation. Biotechnology and Bioengineering 92:416-426.
61. Li, L., Q. Li, L. Rohlin, U. Kim, K. Salmon, T. Rejtar, R. P. Gunsalus, B. L. Karger, and J. G. Ferry. 2007. Quantitative proteomic and microarray analysis of the archaeon Methanosarcina acetivorans grown with acetate versus methanol. J. Proteome. Res. 6:759-771.
62. Li, Q., L. Li, T. Rejtar, D. J. Lessner, B. L. Karger, and J. G. Ferry. 2006. Electron transport in the pathway of acetate conversion to methane in the marine archaeon Methanosarcina acetivorans. Journal of Bacteriology 188:702-710.
63. Liu, J. S., I. W. Marison, and U. von Stockar. 2001. Microbial growth by a net heat up-take: A calorimetric and thermodynamic study on acetotrophic methanogenesis by Methanosarcina barkeri. Biotechnol. Bioeng. 75:170-180.
64. Ma, Q., Z. Yang, M. Pu, W. Peti, and T. K. Wood. 2011. Engineering a novel c-di-GMP-binding protein for biofilm dispersal. Environ. Microbiol. 13:631-642.
65. Ma, Q., G. Zhang, and T. K. Wood. 2011. Escherichia coli BdcA controls biofilm dispersal in Pseudomonas aeruginosa and Rhizobium meliloti. BMC Research News 4:447.
66. MacAuley, S. R., S. A. Zimmerman, E. E. Apolinario, C. Evilia, Y. Hou, J. G. Ferry, and K. R. Sowers. 2009. The archetype g-class carbonic anhydrase (Cam) contains iron when synthesized in vivo. Biochemistry 48:817-819.
67. Maupin-Furlow, J., and J. G. Ferry. 1996. Characterization of the cdhD and cdhE genes encoding subunits of the corrinoid iron-sulfur enzyme of the CO dehydrogenase complex from Methanosarcina thermophila. Journal of Bacteriology 178:340-346.
68. Miller, L. D., J. J. Mosher, A. Venkateswaran, Z. K. Yang, A. V. Palumbo, T. J. Phelps, M. Podar, C. W. Schadt, and M. Keller. 2010. Establishment and metabolic analysis of a model microbial community for understanding trophic and electron accepting interactions of subsurface anaerobic environments. BMC Microbiol 10:149.
69. Moran, J. J., C. H. House, K. H. Freeman, and J. G. Ferry. 2005. Trace methane oxidation studied in several Euryarchaeota under diverse conditions. Archaea 1:303-309.
70. Murakami, E., U. Deppenmeier, and S. W. Ragsdale. 2001. Characterization of the intramolecular electron transfer pathway from 2-hydroxyphenazine to the heterodisulfide reductase from Methanosarcina thermophila. J. Biol. Chem. 276:2432-2439.
71. Murakami, E., and S. W. Ragsdale. 2000. Evidence for intersubunit communication during acetyl-CoA cleavage by the multienzyme CO dehydrogenase/acetyl-CoA synthase complex from Methanosarcina thermophila. Evidence that the beta subunit catalyzes C—C and C—S bond cleavage. J. Biol. Chem. 275:4699-4707.
72. Pantazes, R. J., and C. D. Maranas. 2010. OptCDR: a general computational method for the design of antibody complementarity determining regions for targeted epitope binding. Protein Eng Des Sel 23:849-858.
73. Proposal_Announcement.
74. Ranganathan, S., P. F. Suthers, and C. D. Maranas. 2010. OptForce: an optimization procedure for identifying all genetic manipulations leading to targeted overproductions. PLoS Comput Biol 6:e1000744.
75. Raybuck, S. A., S. E. Ramer, D. R. Abbanat, J. W. Peters, W. H. Orme-Johnson, J. G. Ferry, and C. T. Walsh. 1991. Demonstration of carbon-carbon bond cleavage of acetyl coenzyme A by using isotopic exchange catalyzed by the CO dehydrogenase complex from acetate-grown Methanosarcina thermophila. J Bacteriol 173:929-932.
76. Reichlen, M. J., K. S. Murakami, and J. G. Ferry. 2010. Functional analysis of the three TBP homologs in Methanosarcina acetivorans. Journal of Bacteriology 192:1511-1517.
77. Reichlen, M. J., V. R. Vepachedu, J. C. Murrell, and J. G. Ferry. 2012. MreA functions in the global regulation of methanogenic pathways in Methanosarcina acetivorans. mBio 3:e00189-00112.
78. Rother, M., and W. W. Metcalf. 2004. Anaerobic growth of Methanosarcina acetivorans C2A on carbon monoxide: an unusual way of life for a methanogenic archaeon. Proc. Natl. Acad. Sci. USA 101:16929-16934.
79. Rui, L., L. Cao, W. Chen, K. F. Reardon, and T. K. Wood. 2004. Active Site Engineering of the Epoxide Hydrolase from Agrobacterium radiobacter AD1 to Enhance Aerobic Mineralization of cis-1,2-Dichloroethylene in Cells Expressing an Evolved Toluene ortho-Monooxygenase. Journal of Biological Chemistry 279:46810-46817.
80. Rui, L., L. Cao, W. Chen, K. F. Reardon, and T. K. Wood. 2005. Protein Engineering of Epoxide Hydrolase from Agrobacterium radiobacter AD1 for Enhanced Activity and Enantioselective Production of (R)-1-Phenylethane-1,2-Diol. Applied and Environmental Microbiology 71:3995-4003.
81. Rui, L., Y.-M. Kwon, A. Fishman, K. F. Reardon, and T. K. Wood. 2004. Saturation mutagenesis of toluene ortho-monooxygenase of Burkholderia cepacia G4 for enhanced 1-naphthol synthesis and chloroform degradation. Appl. Environ. Microbiol. 70:3246-3252.
82. Rui, L., K. F. Reardon, and T. K. Wood. 2005. Protein engineering of toluene ortho-monooxygenase of Burkholderia cepacia G4 for regiospecific hydroxylation of indole to form various indigoid compounds. Applied Microbiology and Biotechnology 66:422-429.
83. Saha, R., P. F. Suthers, and C. D. Maranas. 2011. Zea mays iRS 1563: a comprehensive genome-scale metabolic reconstruction of maize metabolism. PLoS One 6:e21784.
84. Saha, R., A. T. Verseput, B. M. Berla, T. J. Mueller, H. B. Pakrasi, and C. D. Maranas. 2012. Reconstruction and comparison of the metabolic potential of cyanobacteria Cyanothece sp. ATCC 51142 and Synechocystis sp. PCC 6803. PLoS One 7:e48285.
85. Sakamoto, T., J. M. Joern, A. Arisawa, and F. H. Arnold. 2001. Laboratory evolution of toluene dioxygenase to accept 4-picoline as a substrate. Applied and Environmental Microbiology 67:3882-3887.
86. Satish Kumar, V., M. S. Dasika, and C. D. Maranas. 2007. Optimization based automated curation of metabolic reconstructions. BMC Bioinformatics 8:212.
87. Satish Kumar, V., J. G. Ferry, and C. D. Maranas. 2011. Metabolic reconstruction of the archaeon methanogen Methanosarcina acetivorans. BMC Syst. Biol. 5:28.
88. Satish Kumar, V., J. G. Ferry, and C. D. Maranas. 2011. Metabolic reconstruction of the archaeon methanogen Methanosarcina Acetivorans. BMC Syst Biol 5:28.
89. Scheller, S., M. Goenrich, R. Boecher, R. K. Thauer, and B. Jaun. 2010. The key nickel enzyme of methanogenesis catalyses the anaerobic oxidation of methane. Nature 465:606-608.
90. Schmitz, S., M. Martinez-Julvez, C. Gomez-Moreno, and H. Bohme. 1998. Interaction of positively charged amino acid residues of recombinant, cyanobacterial ferredoxin:NADP+ reductase with ferredoxin probed by site directed mutagenesis. Biochim. Biophys. Acta 1363:85-93.
91. Shima, S., M. Krueger, T. Weinert, U. Demmer, J. Kahnt, R. K. Thauer, and U. Ermler. 2012. Structure of a methyl-coenzyme M reductase from Black Sea mats that oxidize methane anaerobically. Nature 481:98-101.
92. Sowers, K. R., S. F. Baron, and J. G. Ferry. 1984. Methanosarcina acetivorans sp. nov., an acetotrophic methane-producing bacterium isolated from marine sediments. Appl. Environ. Microbiol. 47:971-978.
93. Spring, S., A. Lapidus, M. Schröder, D. Gleim, D. Sims, L. Meincke, T. Glavina Del Rio, H. Tice, A. Copeland, J.-F. Cheng, S. Lucas, F. Chen, M. Nolan, D. Bruce, L. Goodwin, S. Pitluck, N. Ivanova, K. Mavromatis, N. Mikhailova, A. Pati, A. Chen, K. Palaniappan, M. Land, L. Hauser, Y.-J. Chang, C. D. Jeffries, P. Chain, E. Saunders, T. Brettin, J. C. Detter, M. Göker, J. Bristow, J. A. Eisen, V. Markowitz, P. Hugenholtz, N. C. Kyrpides, H.-P. Klenk, and C. Han. 2009. Complete genome sequence of Desulfotomaculum acetoxidans type strain (5575 T). Standards in Genomic Sciences 1:242-253.
94. Stemmer, W. P. C. 1994. DNA Shuffling by Random Fragmentation and Reassembly: In vitro Recombination for Molecular Evolution. Proceedings of the National Academy of Sciences 91:10747-10751.
95. Stemmer, W. P. C. 1994. DNA shuffling by random fragmentation and reassembly: in vitro recombination for molecular evolution. Proc. Natl. Acad. Sci. U.S. A. 91:10747-10751.
96. Stemmer, W. P. C. 1994. Rapid evolution of a protein in vitro by DNA shuffling. Nature (London, United Kingdom) 370:389-391.
97. Suthers, P. F., M. S. Dasika, V. S. Kumar, G. Denisov, J. I. Glass, and C. D. Maranas. 2009. A genome-scale metabolic reconstruction of Mycoplasma genitalium, iPS189. PLoS Comput Biol 5:e1000285.
98. Tao, Y., W. E. Bentley, and T. K. Wood. 2005. Phenol and 2-naphthol production by toluene 4-monooxygenases using an aqueous/dioctyl phthalate system. Applied Microbiology and Biotechnology 68:614-621.
99. Tao, Y., W. E. Bentley, and T. K. Wood. 2005. Regiospecific Oxidation of naphthalene and fluorene by toluene monooxygenases and engineered toluene 4-monooxygenase of Pseudomonas mendocina KR1. Biotechnology and Bioengineering 90:85-94.
100. Tao, Y., A. Fishman, W. E. Bentley, and T. K. Wood. 2004. Altering Toluene 4-monooxygenase by Active-Site Engineering for the Synthesis of 3-Methoxycatechol, Methoxyhydroquinone, and Methylhydroquinone. Journal of Bacteriology 186:4705-4713.
101. Tao, Y., A. Fishman, W. E. Bentley, and T. K. Wood. 2004. Oxidation of benzene to phenol, catechol, and 1,2,3-trihydroxybenzene by toluene 4-monooxygenase of Pseudomonas mendocina KR1 and toluene 3-monooxygenase of Ralstonia pickettii PKO1. Applied and Environmental Microbiology 70:3814-3820.
102. Terlesky, K. C., and J. G. Ferry. 1988. Ferredoxin requirement for electron transport from the carbon monoxide dehydrogenase complex to a membrane-bound hydrogenase in acetate-grown Methanosarcina thermophila. J. Biol. Chem. 263:4075-4079.
103. Terlesky, K. C., M. J. K. Nelson, and J. G. Ferry. 1986. Isolation of an enzyme complex with carbon monoxide dehydrogenase activity containing a corrinoid and nickel from acetate-grown Methanosarcina thermophila. J. Bacteriol. 168:1053-1058.
104. Thauer, R. K., and S. Shima. 2007. Methane as fuel for anaerobic microorganisms. Ann. N. Y. Acad. Sci. 1125:158-170.
105. Tripp, B. C., C. B. Bell, F. Cruz, C. Krebs, and J. G. Ferry. 2004. A role for iron in an ancient carbonic anhydrase. J. Biol. Chem. 279:6683-6687.
106. Vardar, G., Y. Tao, J. Lee, and T. K. Wood. 2005. Alanine 101 and Alanine 110 of the Alpha Subunit of Pseudomonas stutzeri OX1 Toluene-o-Xylene Monooxygenase Influence the Regiospecific Oxidation of Aromatics. Biotechnology and Bioengineering 92:652-658.
107. Vardar, G., and T. K. Wood. 2005. Alpha-Subunit Positions Methionine 180 and Glutamate 214 of Pseudomonas stutzeri OX1 Toluene-o-Xylene Monooxygenase Influence Catalysis. J. Bacteriol. 187:1511-1514.
108. Vardar, G., and T. K. Wood. 2005. Protein Engineering of Toluene-o-Xylene Monooxygenase from Pseudomonas stutzeri OX1 for Enhanced Chlorinated Ethene Degradation and o-Xylene Oxidation. Applied Microbiology and Biotechnology 68:510-517.
109. Vardar, G., and T. K. Wood. 2005. Protein Engineering of Toluene-o-Xylene Monooxygenase from Pseudomonas stutzeri OX1 for Oxidizing Nitrobenzene to 3-Nitrocatechol, 4-Nitrocatehcol, and Nitrohydroquinone. Journal of Biotechnology 115:145-156.
110. Vardar, G., and T. K. Wood. 2004. Protein Engineering of Toluene-o-Xylene Monooxygenase from Pseudomonas stutzeri OX1 for Synthesizing 4-Methylresorcinol, Methylhydroquinone, and Pyrogallol. Applied and Environmental Microbiology 70:3253-3262.
111. Vepachedu, V. R., and J. G. Ferry. 2012. Role of the fused corrinoid/methyl transfer protein CmtA during CO-dependent growth of Methanosarcina acetivorans. J. Bacteriol. 194:4161-4168.
112. Wang, M., J. F. Tomb, and J. G. Ferry. 2011. Electron transport in acetate-grown Methanosarcina acetivorans. BMC Microbiol. 11:165.
113. Wood, T., K., posting date. [Online.]
114. Zhang, J.-H., G. Dawes, and W. P. C. Stemmer. 1997. Directed evolution of a fucosidase from a galactosidase by DNA shuffling and screening. Proc. Natl. Acad. Sci. U.S.A. 94:4504-4509.
115. Zhang, X.-S., R. García Contreras, and T. K. Wood. 2007. YcfR (BhsA) influences Escherichia coli biofilm formation through stress response and surface hydrophobicity. J. Bacteriol. 189:3051-3062.
116. Zhao, H., and F. H. Arnold. 1997. Optimization of DNA shuffling for high fidelity recombination. Nucleic Acids Research 25:1307-1308.
117. Zomorrodi, A. R., and C. D. Maranas. 2010. Improving the iMM904 S. cerevisiae metabolic model using essentiality and synthetic lethality data. BMC Syst Biol 4:178.
118. Zomorrodi, A. R., and C. D. Maranas. 2012. OptCom: A Multi-Level Optimization Framework for the Metabolic Modeling and Analysis of Microbial Communities. PLoS Comput Biol 8:e1002363.

What is claimed is:

1. An engineered microbe strain that converts methane to a biofuel.

2. The engineered microbe strain of claim 1 wherein the engineered microbe strain is Methanosarcina acetivorans with an engineered reversal of the natural pathway for acetate conversion to methane.

3. An engineered microbe strain that reverses a methylreductase to capture methane.

4. A system comprising:
a. a first engineered organism comprising a methlyreductase capable of capturing methane to produce an intermediate;
b. a second engineered organism that converts the intermediate to a biofuel.

5. The system of claim 4 further comprising an engineered enzyme that converts the methane to the intermediate in the first engineered organism.

6. The system of claim 4 wherein the biofuel is ethanol.

7. The system of claim 6 wherein the intermediate is acetate.

8. The system of claim 4 wherein the biofuel is butanol.

9. A method, comprising:
providing a modified first organism, wherein said modified first organism comprises a methlyreductase capable of capturing methane and producing an intermediate.

10. The method of claim 9 further comprising providing a second organism that converts the intermediate to a biofuel.

11. The method of claim 10 further comprising using the modified first organism to capture the methane.

12. The method of claim 11 further comprising using the modified second organism to convert the intermediate to the biofuel.

13. The method of claim 12 wherein the second organism is a modified organism.

14. The method of claim 13 wherein the biofuel is ethanol.

15. The method of claim 13 wherein the biofuel is butanol.

16. The method of claim 9 wherein the intermediate is acetate.

17. The method of claim 9 wherein the first organism is Methanosarcina acetivorans.
